(19) United States
(12) Patent Application Publication
(10) Pub. No.: US 2026/0350385 A1
(43) Pub. Date: Nov. 5, 2026

(54) SYSTEM AND METHOD FOR IMPROVED SCREENING OF SUBJECTS

(71) Applicant: Joonsung Chyung, Lexington, MA (US)

(72) Inventor: Joonsung Chyung, Lexington, MA (US)

(21) Appl. No.: 19/320,260

(22) Filed: Sep. 5, 2025

Related U.S. Application Data
(60) Provisional application No. 63/691,672, filed on Sep. 6, 2024.
(60) Provisional application No. 63/736,363, filed on Dec. 19, 2024.
(60) Provisional application No. 63/789,106, filed on Apr. 15, 2025.
(60) Provisional application No. 63/822,550, filed on Jun. 12, 2025.

(51) Int. Cl. G06N 3/08; G16H 10/40

(52) U.S. Cl. CPC G06N 3/08; G16H 10/40

(57) ABSTRACT

Systems, methods, and computer-readable storage media for generating tests offering improved screening of subjects. A system can receive historical data associated with multiple variables and identify a cutoff value for each variable. The system can also generate a neural network using the historical data. The system can then generate, using the cutoff values and the neural network, one or more combination tests using multiple variables and the cutoff values for those variables. When a new sample is received, the system can then execute the one or more combination tests using the new sample's data, resulting in a prediction regarding the new sample. The one or more combination tests can then be tested and combined to form an ensemble test.

### PRIORITY

This application claims priority to U.S. provisional patent application No. 63/691,672, filed Sep. 6, 2024, to U.S. provisional patent application No. 63/736,363, filed Dec. 19, 2024, to U.S. provisional patent application No. 63/789,106, filed Apr. 15, 2025, and to 63/822,550, filed Jun. 12, 2025, the contents of each of which are incorporated herein in their entirety.

### BACKGROUND

### 1. Technical Field

The present disclosure relates to a system that generates tests offering improved screening of subjects.

### 2. Introduction

Anomaly identification is important in predicting outcomes or events that have significant consequences, which can be adverse or desirable. Prevention measures or high-intensity screening for these occurrences are a commonly taken approach. However, selection of people or cases to undergo such action is often imprecise, or in some situations not feasible, and may be intrusive, burdensome, costly, as well as resource intensive.

### SUMMARY

Additional features and advantages of the disclosure will be set forth in the description that follows, and in part will be understood from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

Disclosed are systems, methods, and non-transitory computer-readable storage media which provide a technical solution to the technical problem described. A method for performing the concepts disclosed herein can include: receiving, at a computer system, historical data, the historical data comprising: a plurality of known samples; a binary outcome of each sample in the plurality of known samples; and variable data for each sample in the plurality of known samples, wherein the variable data comprises results for a plurality of variables; identifying, via at least one processor of the computer system based on the historical data, a cutoff value for each variable in the plurality of variables, resulting in a plurality of cutoff values; generating, via the at least one processor using the plurality of cutoff values, at least one combination test; receiving, at the computer system, data associated a new sample; and generating, via the at least one processor using the at least one combination test, a prediction of the new sample.

A system configured to perform the concepts disclosed herein can include: at least one processor; and a non-transitory computer-readable storage medium having instructions stored which, when executed by the at least one processor, cause the at least one processor to perform operations comprising: receiving historical data, the historical data comprising: a plurality of known samples; a binary outcome of each sample in the plurality of known samples; and variable data for each sample in the plurality of known samples, wherein the variable data comprises results for a plurality of variables; identifying, based on the historical data, a cutoff value for each variable in the plurality of variables, resulting in a plurality of cutoff values; generating, using the plurality of cutoff values, at least one combination test; receiving data associated a new sample; and generating, using the at least one combination test, a prediction of the new sample.

A non-transitory computer-readable storage medium configured as disclosed herein can have instructions stored which, when executed by at least one processor, cause the at least one processor to perform operations which include: receiving historical data, the historical data comprising: a plurality of known samples; a binary outcome of each sample in the plurality of known samples; and variable data for each sample in the plurality of known samples, wherein the variable data comprises results for a plurality of variables; identifying, based on the historical data, a cutoff value for each variable in the plurality of variables, resulting in a plurality of cutoff values; generating, using the plurality of cutoff values, at least one combination test; receiving data associated a new sample; and generating, using the at least one combination test, a prediction of the new sample.

### BRIEF DESCRIPTION OF THE DRAWINGS

FIG. 1 illustrates an example of the creation of ensemble tests which can be used to filter data;

FIG. 2 illustrates an exemplary process for creating ensemble tests;

FIG. 3 illustrates an example of three datasets being evaluated using processes disclosed herein;

FIG. 4 illustrates an example of a single dataset with a cutoff identified;

FIG. 5 illustrates an example of combination tests;

FIG. 6 illustrates an example of an ensemble test;

FIG. 7 illustrates an example method embodiment;

FIG. 8 illustrates an example of a confirmation test;

FIG. 9 illustrates an exemplary process for creating ensemble tests based upon recursive filtering of the training data;

FIG. 10 illustrates an example method embodiment for test generated from recursive filtering of the training data;

FIG. 11 illustrates an example confirmation method embodiment;

FIG. 12 illustrates an example of filtering based on selection of combination tests; and

FIG. 13 illustrates an example computer system.

### DETAILED DESCRIPTION

Various embodiments of the disclosure are described in detail below. While specific implementations are described, this is done for illustration purposes only. Other components and configurations may be used without parting from the spirit and scope of the disclosure.

As stated above, anomaly identification to predict significant outcomes or events is important, but selection of people or cases requiring certain testing, procedures, or other actions (to prevent, promote, or screen for such occurrences) is very non-precise or unfeasible. This can lead to unnecessary burdens, as most people or cases do not need such actions, and may be harmful, inefficient, intrusive, costly, resource-intensive or impractical.

There exists a need for an improved process to screen people or cases to identify the subset for which certain testing, procedures, or other actions would be appropriate. Currently available screening tests are often suboptimal. For example, they may have excessively high false positive rates or excessively high false negative rates. They may be impractical and require the collection of data that is difficult to access, overly intrusive or even harmful, and/or unreliable. For some type of currently available tests, such as tests derived from machine learning, it is not even clear how those tests work or if they are potentially engaging in demographic discrimination or other forms of bias.

As one example, consider background checks, tests, and other possibly intrusive or burdensome measures for screening prospective customers for receiving insurance or loans. Requiring every person (or case) to undergo extensive testing, vetting, background checks, or other requirements leads to wasting of time and resources (and high cost) to the companies, while imposing unnecessary burden and barriers to prospective customers.

As another example, consider the sending of online advertisements to potential customers. Typically, current ad targeting is still highly imprecise. Many users are sent advertisements, and only a small percentage of users will end up clicking on the advertisements because the advertisements are of interest to only that small group of people. This status quo approach leads to unnecessary cost to the company sending out the advertisements, and is intrusive and burdensome to most users receiving the advertisements.

Yet another example, consider the mass screening tests conducted upon patients, such as colonoscopies or mammograms. Only a small percentage of patients have cancer that would be detected by such tests. As a result, a very large number of individuals must undergo unnecessary testing procedures that introduce burdens and discomforts. Tests such as colonoscopies also have a risk of medical complications for patients. Screening large numbers of patients to detect a small subset of individuals with cancer also leads to unnecessary costs for the health care system.

Systems configured as disclosed herein use historical data to generate tests to screen or otherwise filter future information to predict if a given person or instance will have a certain binary outcome (i.e., yes/no if the outcome will occur or not). While each of the one or more tests can provide a clear binary prediction, they can also be combined together into an ensemble test which, when executed, provides a higher level of accuracy in predicting if the outcome will occur. Moreover, because the overall test is an ensemble of lower tests (described herein as “combination tests”), the system can execute portions of the ensemble test in parallel with one another. If, during the testing, a portion of the ensemble test provides a positive result, the system can (optionally) cease testing, thereby saving computational resources given that the ultimate conclusion (a positive result) has already been reached. In this manner the testing is not only more accurate, but can also be faster (due to the parallel testing) while preserving (when appropriate) computational resources/power.

The availability of such generated tests can significantly reduce the number of people or instances that need to be screened or intervened upon. For example, instead of conducting screening tests on a large number of people to detect a disease or other problem, systems configured as disclosed herein can reduce that number to a smaller subset needed for testing while still ensuring that almost all positive cases would be detected, despite the lower number of screenings performed. As another example, instead of sending advertisements to a large number of prospective customers, systems configured as disclosed herein can reduce the number of people for which advertisements should be sent to, while still reaching the customers who would have positively responded to the advertisement.

There are other favorable features of tests generated from this system, including advantages over current methods (including conventional approaches as well as machine learning-based approaches). Examples of such additional features include but are not limited to: 1) the ability to use a broader range of different types of variables, including variables that would otherwise be ignored by status quo methods; 2) the explainability of generated tests; 3) the ability to attain high sensitivities for datasets that only have a small number of positive samples; and 4) the ability to fine tune the desired sensitivity and specificity of a test emerging from the system. To illustrate, systems configured as disclosed herein can generate tests that employ a broader set of different types of variables, including variables that may otherwise be commonly ignored (such as variables with Receiver Operating Characteristic (ROC) curves that may otherwise be considered poor or suboptimal) by status quo methods. Many variables may have useful information (and may be used by systems configured as disclosed herein), while conventional methods may ignore or not use those variables. Also, commonly used machine learning algorithms or other diagnostic approaches may rely upon variables for which there is incomplete or missing data, or for which it is not practical to obtain the data. In contrast, systems and methods configured as disclosed herein can make use of variables for which there may be more complete datasets and easier-to-obtain data.

In addition, tests generated by the system are explainable. This allows the user to have a clear and explicit understanding of how a particular test generated by the system works. In addition, the processes described herein clearly identify which variables (along with their respective cutoff values) and combinations are used for a particular test. In this way the user can ensure that such variables have adequate and reliable data collection, and can also ensure that unintentional discrimination based upon inappropriate or inequitable factors is avoided. Such explainability stands in contrast to tests commonly derived from status quo machine learning methods, as it is often unknown or unclear how such machine learning-derived tests actually work, such as which variables those machine learning-derived tests use or how they use such variables.

Moreover, tests generated by the system as disclosed herein can attain high sensitivities (without significantly compromising specificity) for datasets that only have a small number of positive samples. The system can, for example, produce tests from highly imbalanced datasets. This is of importance, as screening may sometimes involve “finding a needle in a haystack” due to the very low rate of samples/instances/individuals that have the binary outcome of interest within the broader population. Maintaining high specificity is important in such contexts, or else such tests can lead to an excessive rate of false positives.

Additionally, the system is easily tunable in that the user can set up the sensitivity and specificity profile for a particular test that they want to generate for their specific use case scenario (i.e., minimum sensitivity and minimum specificity can both be specified by the user).

Another feature offered by the system is the ability to identify profiles of cases that are of particular interest. Due to the explainability of tests generated from this system, one can identify combinations of positive variables that are associated with a disproportionately high PPV for an outcome of interest. For example, one could identify a constellation of positive variables related to how a machine is used or the machine's status which is associated with a high likelihood of the machine breaking down. Then, one can proactively take measures to intervene pre-emptively to prevent the occurrence of such parameters in the first place. Or, as another example, one could identify a particular profile of customers who are highly likely to purchase a product. One could then study that particular customer profile to optimize the design of a product to appeal to that customer segment or to expand marketing outreach to that customer segment. There are many other real-world opportunities from case profile identification, such as disease prevention, accident prevention, ad targeting, and successful product identification.

In addition, the system can generate screening tests that need only minimal computation time to execute them. In other words, once a test is generated from the system, the inference time needed to run it upon a new sample can be very low.

As a more detailed description of the system, the system generates a test for a given specific use case to predict a particular binary outcome (e.g., predict if a patient has colon cancer or not, predict if the stock price for a given company will go up by a specified percentage or not, predict if a customer will purchase a product or not, predict if a case represents credit card fraud or not, etc.) based upon using a historical dataset (training dataset).

Once generated, each test looks at combinations of variables for a given new sample (e.g., data across various variables for an individual patient, customer, or case). For example, with breast cancer screening, some of the variables under consideration may include sex, age, if the individual smokes, family history of breast cancer, etc.

For each variable, there is a specified cutoff value which, if the numerical value in the sample exceeds that threshold (in some cases, the value must be greater than that cutoff, whereas in other cases, the value must be less than that cutoff), then the variable is considered positive. A test which considers if multiple variables meet their respective cutoff values is a “combination test.” In a combination test, if every variable within the combination test is positive, then the output of the combination test will be considered positive.

The system can further combine the combination tests together, creating an “ensemble” of combination test, also referred to as an ensemble test. If at least one combination from within the ensemble is positive, then the overall test result is considered positive, thus predicting that the binary outcome of interest will occur for that particular sample (e.g., patient, customer, or case).

As an illustrative example, consider a test that is trying to predict if a patient may have colon cancer. Each patient has ten different variables of data (designated in this illustrative example as variables a, b, c, d, e, f, g, h, i, and j). The overall/ensemble test uses seven different combinations of variables: combination #1 (a, c), combination #2 (a, f, i), combination #3 (b, e, h, i), combination #4 (d, f, j), combination #5 (e, g, I), combination #6 (e, f, h, j), and combination #7 (g, h). For each individual variable, there is a cutoff value that has been selected (based upon the system's analysis of the historical dataset).

When a new patient is being evaluated by the system, the system executes the overall/ensemble test, with the ensemble test identifying the numerical values for each of the ten different variables. If the numerical value for a given variable meets the threshold (i.e., exceeds the identified cutoff value), then that individual variable is considered positive for that patient. However, a positive variable is not enough for the patient's test result to be considered positive. In order for the patient's test result to be determined as positive (i.e., predicting that the patient has colon cancer in this example), at least one of the combination tests (among the 7 combinations in the ensemble) must be positive for that patient. In order for a combination to be considered positive, all of the individual variables within that combination must be positive (e.g., for combination #1 above, variables a, c, and g must each be positive).

Sensitivity is the ability of a test to identify people with a given condition as positive, while specificity is the ability of a test to identify people without the condition as negative. As a conceptual basis for how the systems and methods outlined herein offer improved sensitivity and specificity, consider the limitations of relying upon a single individual variable to predict whether a sample will be positive for the binary outcome of interest. It is rare for testing based upon individual variables to offer both high sensitivity and high specificity. Instead, there is commonly a trade-off encountered in that use of a cutoff value (to predict the binary outcome) that yields very high sensitivity will be associated with a low specificity and vice versa.

In contrast, combination tests (including the overall/ensemble test) generated by the system do not rely upon a single variable and instead utilize multiple variables that are bundled together as a combination. More specifically, in order for a combination (of variables) to be considered positive and thus predict that the binary outcome of interest will occur, every single variable within that combination must be positive. By requiring that multiple variables are positive and not just one, the false positive rate will be substantially diminished (thereby improving specificity), as it is very unlikely that a sample with multiple positive variables will not be truly positive for the binary outcome. To further minimize the false positive rate overall, each variable within the combination has a cutoff value selected that has a relatively low false positive rate (even if it also yields a low true positive rate). Through these two measures, the stringency of the testing minimizes the occurrence of false positive predictions.

In circumstances where only one combination of variables is used, this would likely reduce the true positive rate of the testing because there may be samples (with true positive outcomes) that do not have the associated occurrence of that positive combination. To address this issue and maximize the true positive rate, systems configured as disclosed herein do not rely upon the presence of a single combination. Instead, the system generates an ensemble test (i.e., a collection of combination tests) for which if any combination (even if just a single combination) within that ensemble test is positive, then the overall/ensemble test predicts a positive binary outcome. Thus, through these various elements in the approach, systems configured as disclosed herein can simultaneously offer very high sensitivity and very high specificity.

To generate the combination tests and overall/ensemble test (both of which will be customized to whichever use case scenario is presented to the system), the user first uploads a “training dataset”. This dataset, or historical dataset, is encompassed by historic data for multiple samples/individuals/situations, where the historical data has, for each sample/individual/situation: (1) a binary value indicating if the sample had positive/negative results (e.g., did the person open the advertisement, or not open the advertisement; did the individual actually have cancer, or did they not have cancer); and (2) variables associated with the sample (e.g., in medical examples the variable data could include an individual's age, gender, age at which family member had cancer, blood pressure, numbers of times per week that red meat is eaten, number of times per day that fruits and vegetables are eaten, minutes of sitting per day, number of times per week that have constipation, number of times per year that have diarrhea, previous medical history, etc.; in advertising examples the variable data could include an individual's age, gender, number of people living in their household, population of the town in which they reside, income, web history, etc.). The historical data may also include a previous prediction regarding whether the results in question (e.g., a prediction that they would test positive, or would test negative). The variables associated with the sample usually are continuous (i.e., a numerical value) but may be discrete (e.g., binary, such as yes or no, or multiple choice) in some instances.

The system analyzes the historical dataset and then generates, for each variable, a Receiver Operating Characteristic (ROC) curve. This curve plots the various true positive rates against the associated false positive rates for various and different cutoff values used for the given variable. To produce such a curve for each variable, the system will try out various cutoff values for the given variable and then evaluate all samples across the historical dataset to determine what percentage of true and false positives would have been predicted for each of the different cutoff values. Thus, for each different possible cutoff value (for the given variable), the system will compute the true positive and false positive rate, thereby enabling the generation of an ROC curve. At each point along this curve, the system will compute the ratio of true positive rate (TPR) to false positive rate (FPR), i.e. the TPR/FPR ratio.

For each variable, the system surveys points along the ROC curve to identify the particular point having the highest TPR/FPR ratio, along with an additional requirement that the TPR is >0.05 (or other value if the user would like to specify a different lower limit for the TPR for the cutoff value selection). The value of the variable along the ROC curve that yields this TPR/FPR ratio is identified as the cutoff value that should be selected for that particular variable.

When a new sample is received, the system can compare data for that new sample against the cutoff values for each variable. If the sample data for a given variable meets the cutoff value for that variable, that indicates that this variable (for that particular sample) is positive, whereas if the sample data does not meet the cutoff value, that indicates that this variable is negative (hence a binary result). In some instances, the variable is considered positive if the variable for a given sample has a numerical value that is greater than the selected cutoff value. In other instances, the variable is considered positive if the variable for a given sample has a numerical value that is lower than the selected cutoff value.

After determining the cutoff value for each individual variable based upon the above analysis of the historical dataset, the system then generates all possible different combinations of individual variables, resulting in combination tests. The maximum number of variables that can be included in a combination test can be specified by the user, since it may be too computationally expensive (i.e., overload the computing power or take too long for the computer to run) if the number of variables is too high. For example, a user can specify that up to 5 variables can be included in a combination test. Then, the system will generate all possible combinations of variables. For example, if there are 20 total variables in the historical dataset, then the system will generate combination tests using all possible combinations of variables drawn from the pool of 20 variables, with up to 5 variables per combination test. (Combination tests in this example can consist of 1, 2, 3, 4, or 5 variables).

For each combination test that has been generated, the combination test result for a given sample will be considered positive if (and only if) every individual variable within that combination test is positive (i.e., based upon if the cutoff threshold is exceeded).

Next, the user decides upon a minimum sensitivity and specificity that they are seeking for the test to be generated by the system. This decision will vary upon the particular use case of interest. For example, for some tests, the user may wish to seek a very high sensitivity and be willing to have the test have a relatively lower specificity in order to achieve this aim. For other tests, the opposite may be the case for the user's goals for the test to be generated. There are also other profiles for sensitivity and specificity that a user may wish for the test.

Using the equation below, the minimum positive predictive value (minPPV) that the overall test should have is calculated:

$$min PPV = \frac{totalP \cdot min TPR}{totalP \cdot min TPR + ( 1 - min TNR ) \cdot totalN} \qquad Equation 1$$

Where:
minPPV is the minimum positive predictive value of the final test;
totalP is the total number of positives among the samples;
minTPR is the minimum true positive rate (sensitivity) specified by the user for the test. Please note that this minTPR is different from the 0.05 (or other user-specified value) lower limit requirement for TPR in selecting the cutoff value for each variable. Instead, this minTPR is referring to the value the user specifies as the minimum sensitivity that the user would like for the overall test (i.e., the ensemble test) that is to be generated by the system;
minTNR is the minimum true negative rate (specificity) specified by the user for the test; and
totalN is the total number of negatives among the samples.

Consider the following example. A user wishes to use the system disclosed herein to generate a screening test that will predict if a customer will purchase a particular product. Previously, the user sent online advertisements to a non-targeted audience of customers, leading to 0.1% yield in customer purchases. In using the disclosed system, the user intends to have a higher success rate in predicting which customers will purchase the product. The user can use the previous success rate to guide the targeted delivery of advertisements to customers more likely to purchase the product. For this new screening, the user decides to specify a minimum sensitivity and minimum specificity, leading to a minPPV of 1%. The user's decision to select this minPPV for the test is sensible because, compared to a 0.1% yield for sending advertisements to a less targeted audience of customers, use of the system would enable a 10-fold lower number of advertisements to be sent.

After the user specifies what the minPPV should be for the overall/ensemble test to be generated, the system will use the historical dataset to calculate the PPV for each of the possible combination tests that had been generated. This PPV is calculated for each combination test by treating each combination test as if it were a standalone test, then evaluating that combination test's ability to correctly predict true positives by surveying every sample within the historical dataset. As a reminder, a given combination test is considered to predict a positive outcome for a given sample if each and every variable within that combination is positive, meaning that each individual variable in that combination test exceeds its designated cutoff value.

Then, the system will identify and select only the subset of combination tests that have a PPV that is at least as high as the specified minPPV.

Next, the system will assemble together the ensemble test (i.e., the collection of variable combination tests) to be used. Among the combinations that have a PPV at least as high as the specified minPPV, the system will use the historical dataset to calculate the True Positive Rate (TPR) for each combination test. The combination test offering the highest TPR among the various combination tests will be selected as the first combination test to be included in the overall ensemble test (and thus the overall).

Next, the system will search for a second combination test to be added to the ensemble test. Using the remaining combination tests which have a PPV at least as high as the user-specified minPPV, the system will generate every possible pairing of those remaining combination tests with the first selected combination. Each of these possible combination test pairs will then be treated as if the pair were the actual, final ensemble test to determine which pair provides the best (i.e., most accurate) prediction. Again, the ensemble test is considered to predict the binary outcome of interest as positive if at least one of the combination tests within that ensemble test is positive.

Using the historical dataset, each of these generated combination test pairs are assessed for the ability to correctly predict true positives by surveying every sample within the historical dataset. A TPR and PPV is calculated in this manner for each of the generated combination test pairs. The second combination test that (upon pairing with the first combination test already selected) yields an ensemble test with the highest PPV, while still increasing the ensemble test's TPR, is selected by the system as the second combination test to be included in the ensemble.

This process is then repeated to identify a third combination test to add to the ensemble. The system generates every possible ensemble consisting of a third combination test (from among the remaining combination tests that have a PPV at least as high as the specified minPPV) added to the already selected pair of combination tests. Using the historical dataset, each of these generated ensembles are assessed for the ability to correctly predict true positives by surveying every sample within the historical dataset. A TPR and PPV is calculated in this manner for each of the generated combination test triplets. The third combination test that (upon being assembled with the first two combination tests already selected) yields an ensemble test with the highest PPV (out of the possible ensemble tests), while still increasing the ensemble test's TPR, is selected by the system as the third combination test to be included in the ensemble test.

In this manner, the ensemble can then continue growing, with the system adding one combination test to the ensemble per cycle of expansion. Every possible ensemble (that is increased in size by one combination test more than the prior constructed ensemble) is generated by introducing a new combination test (from among the remaining combinations that each have a PPV greater than the user-specified minPPV). Then the historical dataset is used, as before, to calculate the TPR and PPV for each of the candidate larger ensembles. The new combination test that (upon being assembled with any prior combination(s) already selected) yields an ensemble with the highest PPV, while still increasing the ensemble's TPR, is selected by the system as the next combination to be included in the ensemble.

This cycle of expansion for the ensemble test's size proceeds until the system determines that introducing an additional combination would lead to the PPV of the ensemble test being less than the user-specified minPPV for the overall test.

The size of the overall ensemble test will vary depending upon the particular use case scenario (and its associated training dataset) and the user-specified minPPV that the user is employing. For example, a given ensemble test could include 1, 2, 3, 4, 5, or more than 5 combination tests.

In addition to the inclusion of combination tests within an ensemble test, machine learning model tests can also be included with one or more combination tests in some versions of the ensemble test. Any machine learning model test can be included as long as the test can have its PPV determined from the given historical dataset of interest to the user. Examples of such machine learning model tests include tests based on (but are not limited to) models generated using: logistic regression, random forest, K-nearest neighbors, gradient boosting, neural network (e.g., Artificial Intelligence), etc.

The system evaluates each machine learning model test in the same manner as it does for each combination test to evaluate if it should be included in the ensemble test. It is therefore possible for tests generated from the system to be encompassed by an ensemble comprised of one or more different machine learning model tests, together with one or more different combination tests, in which the sequence of the various machine learning model tests and combination tests within the ensemble can be in any particular order. It is also possible for ensemble tests generated from the system to be comprised only of combination tests (i.e., there may not be any machine learning model tests included in the ultimate ensemble of tests). It is also possible for ensemble tests to be comprised only of machine learning model tests as long as there are two or more machine learning model tests.

For example, the system could be used to generate a test (i.e., an ensemble of tests) that detects when a particular type of machine is at risk of failing. This example ensemble test has its first ensemble member being a random forest algorithm test, the second through fourth ensemble test members being three different combination tests, the fifth ensemble test member being a logistic regression algorithm test, and the sixth ensemble test member being a 4^{th} combination test. As with system-generated tests embodied by ensemble tests made up of only combination tests, this example ensemble test operates by applying the first ensemble member test (in this example, the random forest algorithm test) to see if a given data sample is positive or not upon applying that particular ensemble test member. If the test result is positive, then the overall test is considered positive for that given sample. If the particular ensemble test member returns a negative test result, then the next ensemble test member (in this particular example, a combination test) is applied to see if that test returns a positive or negative test result. As before, if the test result is positive, then the overall test is considered positive for that given sample. If the particular ensemble test member returns a negative test result, then the next ensemble test member is evaluated. This cycle of testing repeats until a positive result is observed for the given sample (in which case the overall test result is positive for that sample) or until all members of the ensemble test have a negative result for the given sample (in which case the overall test result is negative for that sample).

Another example of a system-generated test could be encompassed by an ensemble test in which the first three members are combination tests, the fourth is a gradient boosting algorithm test, and the fifth through tenth members are combination tests.

Yet another example of a system-generated test could be encompassed by an ensemble test in which the first member is a logistic regression test, the second member is a neural network test, and the third and fourth members are combination tests. There are many other possible ensembles that are used for system-generated tests.

The approach to evaluating a given machine learning model (and their associated tests) for inclusion in a particular ensemble test is the same as that for evaluating the various combination tests. As with the previously described approach for test generation, the user specifies what the minPPV should be for the overall/ensemble test to be generated. The system can then use the historical dataset for the particular use case to calculate the PPV for each of the machine learning model tests as well as for all the possible combination tests that had been generated. As with the approach to the combination tests, this PPV is calculated for each machine learning model test by treating each machine learning model test as if it were a standalone test, then evaluating that machine learning model test's ability to correctly predict true positives by surveying every sample within the historical dataset.

The system can then identify and select only the subset of combination tests and/or machine learning model tests that have a PPV that is at least as high as the specified minPPV. If a given machine learning model test (whether it is the default version or a variant of it) offers a PPV that is at least as high as the user-specified minPPV, then this machine learning model test can be included in the pool of candidates for potential inclusion in the ensemble.

Next, the system can assemble the ensemble test (i.e., the collection of combination tests and/or machine learning model tests) to be used. Among the identified combinations and machine learning model tests that have a PPV at least as high as the specified minPPV, the system will use the historical dataset to calculate the TPR for each combination test and each machine learning model test. The combination test or machine learning model test offering the highest TPR among the various identified combination tests and machine learning model tests will be selected as the first member to be included in the overall ensemble test (and thus the overall test).

Next, the system will search for a second member to be added to the ensemble test. Using the remaining combination tests and machine learning model tests which have a PPV at least as high as the user-specified minPPV, the system will generate every possible pairing of those remaining combination tests and machine learning model tests with the first selected ensemble member. Each of these possible ensemble member pairs will then be treated as if the pair were the actual, final ensemble test to determine which pair provides the best (i.e., most accurate) prediction. Again, the ensemble test is considered to predict the binary outcome of interest as positive if at least one of the combination tests and machine learning model tests within that ensemble test is positive.

Using the historical dataset, each of these generated ensemble member pairs are assessed for the ability to correctly predict true positives by surveying every sample within the historical dataset. A TPR and PPV is calculated in this manner for each of the generated ensemble member pairs. The combination test or machine learning model test that (upon pairing with the first combination test or machine learning model test already selected) yields an ensemble test with the highest PPV, while still increasing the ensemble test's TPR, is selected by the system as the second member to be included in the ensemble.

This process is then repeated to identify a third member to add to the ensemble. The system generates every possible ensemble consisting of a third member (from among the remaining combination tests and machine learning model tests that have a PPV at least as high as the specified minPPV) added to the already selected pair of ensemble members. Using the historical dataset, each of these generated ensembles are assessed for the ability to correctly predict true positives by surveying every sample within the historical dataset. A TPR and PPV is calculated in this manner for each of the generated ensemble member triplets. The third ensemble member that (upon being assembled with the first two ensemble members already selected) yields an ensemble test with the highest PPV (out of the possible ensemble tests), while still increasing the ensemble test's TPR, is selected by the system as the third member to be included in the ensemble test.

In this manner, the ensemble can then continue growing, with the system adding one member (i.e., combination test or machine learning model test) to the ensemble per cycle of expansion. This cycle of expansion for the ensemble test's size proceeds until the system determines that introducing an additional member would lead to the PPV of the ensemble test being less than the user-specified minPPV for the overall test.

The size of the overall ensemble test will vary depending upon the particular use case scenario (and its associated training dataset) and the user-specified minPPV that the user is employing. For example, a given ensemble test could include 1, 2, 3, 4, 5, or more than 5 members (i.e., some combination of different combination tests and/or different machine learning model tests).

There are also variations possible for how the ensemble is built. For example, instead of selecting combination tests (and/or machine learning model tests) for the ensemble based upon requiring a minPPV and then choosing the combination test (or machine learning model test) with the highest TPR among the candidates, the system could select combination tests (and/or machine learning model tests) based upon requiring a minimum TPR and then choosing the combination test (or machine learning model test) with the highest PPV among the candidate combinations. Or as another example, the system could select combination tests (and/or machine learning model tests) for the ensemble based upon the greatest sum of PPV and TPR among the candidates or based upon the highest number when multiplying PPV and TPR among the candidates.

In another version of the system, designated here as the “recursive dataset filtering” approach, ensemble construction involves a recursive refining of the historical dataset used for training after each round of ensemble expansion. More specifically, after each round of adding a new combination test or machine learning model test, the historical dataset used to train the system is filtered to exclude any data samples that the work-in-progress ensemble predicts as positive. Then, using this filtered training data, the system repeats the process of identifying an optimal cutoff value for each individual variable. Next, the system creates candidate combination tests and/or machine learning models and evaluates the candidate combinations and machine learning models to identify which combination test or machine learning model test to add to the growing ensemble. For the next round of ensemble expansion, the previously filtered historical dataset is filtered further to exclude any data samples that the latest iteration of the ensemble predicts as positive. Thus, the training dataset keeps being iteratively filtered after each round of ensemble expansion. Through this approach, the system can generate screening tests with enhanced performance.

To provide context and rationale for this recursive dataset filtering approach, it is helpful to first consider the underlying strategy for how the system operates in general and across all its various versions. The general approach for optimizing screening performance is based upon a division of labor strategy for detecting true positives across the full pool. In tackling a dataset, the goal is to successfully catch all, or nearly all, of the true positive samples, while minimizing the rate of false positive predictions. The system can be thought of as essentially partitioning the pool of true positive samples into subsets and assigning the task of detecting a given subset to a particular combination of variables.

To elaborate, the system first produces a large set of candidate combinations for consideration. To minimize the false positive rate for these various combinations, the cutoff values for individual variables are selected to optimize detection yields and the combinations are constructed such that a combination is considered positive if and only if every variable in that combination is positive. The system then surveys the various candidate combinations to determine which combination is best suited for a given task of detecting a particular subset of true positive samples. To drive this decision-making process, the system, in practice, looks at which combination is most “cost-efficient” in detecting the true positives in that subset, except that rather than a budget based upon financial cost, the cost is the rate of false positives. In other words, the system determines which combination is the least expensive in terms of increasing the false positive rate. (Beyond combinations, the system can also use neural networks or machine learning models in the ensemble and assign them to the task of detecting subsets of true positive samples in the dataset. Thus, the screening tools within an ensemble can be combinations, neural networks, or machine learning models.) The resulting ensemble, then, consists of a collection of screening tools (combinations and/or neural networks and/or machine learning models) which each are focused upon detecting a subset of true positives among the full pool of true positives.

Within the ensemble, there may be some overlap between the subsets of true positives detected by the various screening tools. Whenever such an overlap (i.e., redundancy in detecting true positives) between the screening tools occurs, there is unnecessary expenditure of the false positive rate without gaining sensitivity. To minimize, or eliminate, these overlaps, the alternative version of the system employs a recursive dataset filtering approach in constructing the ensemble. Thus, in doing so, this version of the system offers enhanced screening performance.

As a detailed description of this recursive dataset filtering version of the system, this system will produce candidate combination tests in the same manner as the other versions of the system. More specifically, this recursive dataset filtering version of the system will analyze the historical dataset to determine the cutoff value for each individual variable in the same way as before. This system will also create a pool of candidate combination tests in the same manner as before, including the provision that a combination test is considered positive for a given sample if and only if every variable within that combination test is positive (i.e., based upon if the cutoff threshold is exceeded).

Also, as before, the user will decide upon a minimum sensitivity and specificity that they are seeking for the overall test to be generated by the system, leading to a calculation of the minimum PPV. The selection approach of the first combination test to be included in the ensemble will also be the same as before. The system will use the historical dataset to calculate the PPV for each of the candidate combination tests that had been generated. This PPV is calculated for each combination test by treating each combination test as if it were a standalone test, then evaluating that combination test's ability to correctly predict true positives by surveying every sample within the historical dataset. Then, the system will identify and select only the subset of combination tests that have a PPV that is at least as high as the specified minPPV.

However, in this version of the system, the combination test offering the highest PPV (rather than highest TPR) among the various candidate combination tests will be selected as the first combination test to be included in the ensemble for the overall test. Subsequently, the system will modify the historical dataset to exclude any samples predicted by the current, work-in-progress test (which at this stage will entail an ensemble that is only comprised of the first combination test) to be positive. This includes samples that are true positives as well as false positives, as it only matters that the work-in-progress (i.e., still being built) ensemble test predicts a given sample in the historical dataset as positive.

Then, using this filtered historical dataset, the system will re-perform the process of producing a pool of candidate combination tests. For this step, the system will analyze the filtered historical dataset to determine the cutoff value for each individual variable in the same way as before. However, because this training dataset is now different from the original historical dataset due to the filtering step, the cutoff values for each variable may be different from those determined originally. The system will then create a pool of candidate combination tests in the same manner as before, including the provision that a combination test is considered positive for a given sample if and only if every variable within that combination test is positive (i.e., based upon if the cutoff threshold is exceeded). The TPR for each candidate combination test is calculated by trying this combination test upon every sample in the filtered, historical dataset. Based upon a user-specified minimum TPR for all combination tests (which, it should be noted, is distinct from the user-specified minimum TPR for the overall test that in turn is used to calculate the minPPV), only the combination tests offering a TPR of at least that user-specified minimum value will be considered as a candidate for consideration.

The system will then search among this pool of candidate combination tests to decide which combination test should be paired with the first combination test in the ensemble. The system will generate every possible pairing of those candidate combination tests with the first selected combination. Each of these possible combination test pairs will then be treated as if the pair were the actual, final ensemble test to determine which pair provides the best prediction. Again, the ensemble test is considered to predict the binary outcome of interest as positive if at least one of the combination tests within that ensemble test is positive. Using the original training dataset (i.e., the unfiltered historical dataset), the PPV is calculated for each of these generated combination test pairs by surveying every sample within the original, unfiltered training dataset. The second combination test that (upon pairing with the first combination test already selected) yields an ensemble test with the highest PPV (based upon evaluation via the original, unfiltered training dataset), is selected by the system as the second combination test to be included in the ensemble. It is important to note that the calculated TPR for each combination (to assess if it fulfills the user-specified minimum TPR for all combinations) is calculated using the filtered historical dataset, while the calculated PPV for each combination is calculated using the unfiltered, original historical dataset.

Next, the process that was done for selecting the second combination test in the ensemble is repeated in selecting the third combination test for inclusion. The historical dataset that had been filtered to remove any samples predicted as positive by the first combination test is further filtered to remove any samples predicted by the newly constructed, work-in-progress test (which at this stage will now entail an ensemble comprised of two combination tests) to be positive. As with the step for identifying the second combination test for inclusion in the ensemble, the system will re-perform the process of determining cutoff values for each individual variable based upon an analysis of this further filtered historical dataset. Candidate combination tests will be generated using these new cutoff values for the individual variables.

The system will use the further filtered historical dataset to calculate the TPR for those candidates, identify the candidate combinations which have a TPR at least as high as the user-specified minimum TPR, and generate every possible trio of combination tests (comprised of a candidate combination test and the first two already-selected combination tests). Then, using the original training dataset (i.e., the unfiltered historical dataset), each of these generated combination test trios are assessed for the ability to correctly predict true positives by surveying every sample within this dataset. The combination test that (upon being matched up with the first two combination tests already selected) yields an ensemble test with the highest PPV (based upon evaluation using the unfiltered, original training dataset), is selected by the system as the third combination test to be included in the ensemble.

Each subsequent round of selecting an additional combination test for inclusion in the ensemble is conducted in the same manner. In other words, the system will further filter the historical dataset used for training to remove samples predicted as positive by the latest, work-in-progress ensemble test. Then, using this updated training dataset, the system will re-do the variable cutoff determinations and produce candidate combination tests. Next, the system will identify which combination test to add to the growing ensemble based upon identifying which combination test has a TPR greater than the user-specified minimum TPR for all combinations (based upon evaluating the filtered historical dataset) and which will lead to the highest PPV (based upon evaluating the original, unfiltered historical dataset) for the ensemble.

This cycle of expansion for the ensemble test's size proceeds until the system determines that introducing an additional combination would lead to the PPV of the ensemble test being less than the user-specified minPPV for the overall test. The size of the overall ensemble test will vary depending upon the particular use case scenario (and its associated training dataset) and the user-specified minPPV that the user is employing. For example, a given ensemble test could include 1, 2, 3, 4, 5, or more than 5 combination tests.

In this approach to ensemble construction based upon recursive filtering of the training dataset, machine learning model tests can also be included with one or more combination tests in the ensemble. Any machine learning model test can be included as long as that test can have its TPR and PPV determined. Examples of such machine learning model tests include tests based on (but are not limited to) models generated using: logistic regression, random forest, K-nearest neighbors, gradient boosting, neural network, etc. Thus, the ensembles generated by the version of the system using recursive filtering of the training dataset can be comprised of one or more combination tests, together with zero, one, or more machine learning model tests. Or alternatively, ensembles can be comprised of two or more machine learning model tests in the absence of any combination tests.

There are also variations possible for this version of the system that uses recursive filtering of the historical dataset for training. For example, instead of selecting combination tests (and/or machine learning model tests) for the ensemble based upon requiring a minimum TPR and then choosing the combination test (or machine learning model test) that yields the highest PPV for the emerging ensemble, the system could select combination tests (and/or machine learning model tests) based upon requiring a minimum PPV and then choosing the combination test (or machine learning model test) that yields an emerging ensemble with the highest TPR among the candidate combinations. Or as another example, the system could select combination tests (and/or machine learning model tests) for the ensemble based upon the greatest sum of PPV and TPR among the candidates or based upon the highest number when multiplying PPV and TPR among the candidates.

The use of recursive filtering of the training dataset for the system offers multiple advantages and features. For example, this approach can minimize, or eliminate altogether, the overlap between the various screening tools (combination tests or machine learning model tests) within an ensemble in detecting true positives across the full population of true positives. In doing so, this can yield the generation of screening tests with enhanced performance, such as higher sensitivity (recall), specificity, PPV (precision), and/or NPV.

The following are more specific examples of how systems configured as disclosed herein can be used.

Online advertisement targeting: Systems configured as disclosed herein can significantly reduce the number of users for which an advertisement needs to be sent in order to achieve a certain rate of positive responses. For example, instead of sending an online advertisement to 1 million people, the system generates an ensemble test that can identify the subset of those 1 million individuals for which the online advertisement should be sent, thereby increasing the response ratio (i.e., how often people actually click on the advertisement, or buy something based on the advertisement) for the advertisements. As a specific use case example, the system can evaluate a dataset of people who would otherwise be targeted to receive a specific online advertisement. The system would then develop a specific test (i.e., involving an ensemble of variable combinations, as process described above) that would identify the subset of such people who are at higher likelihood of clicking on the online advertisement. In doing so, the advertisement could be sent to only a smaller subset of the people (i.e., those likely to positively respond to the advertisement), without missing those who would likely have clicked on the advertisement with the non-precise, old advertising approach. In some configurations, the system itself, upon identifying/determining the smaller subset of people, will directly send out the online advertisement across a network (e.g., the Internet), thereby reducing bandwidth/networking requirements to reach the same group of people who would likely have clicked on the advertisement using the old advertising approach. In such configurations, the system may have access to a database containing one or more of the advertisements, enabling the system to market to the identified group of individuals in a manner which saves bandwidth (i.e., a technological improvement) while achieving the same result (i.e., reaching those individuals most likely to click on the advertisement).

Product and service recommendation engines: Systems configured as disclosed herein can significantly improve the customization of product and service recommendation to customers. To illustrate, the system generates an ensemble test that can identify the subset of customers who would be very interested in a given product or service and thus highly likely to purchase or use this offered product or service. In some configurations, the system itself, upon identifying/determining the smaller subset of people who would be highly interested in a particular product, will directly email the product offering to the select subgroup of people or visually display the product option on a mobile webpage, computer webpage, or TV screen as an offering to the people to consider using or purchasing. In doing so, the electronic transmission and display of the product information will only be sent to a subset of people rather than the full population of prospective customers. Thus, it represents a technological improvement, as it reduces the consumption of computational capacity and bandwidth by the website, mobile device, and/or data center, as while still achieving the same result (i.e., reaching those individuals most likely to purchase or use the given product). In another configuration, after identifying a subset of people who would be highly interested in purchasing or using a particular product or service, the system itself, as part of a wearable device or augmented reality device (such as an augmented reality eyeglasses device), will display a notification to such individuals whenever they are in the vicinity of a store or restaurant offering the particular product of interest. Such a product of interest could be a particular type of clothing, toy, video game, furniture, artwork, or other physical good, and examples of services of interest could be a particular food dish, type of haircut, or other service. Thus, the digital display to notify the customer of the nearby presence of a product or service of interest will only be activated in the augmented reality device of a select subset of people rather than all users of that device. Thus, it represents a technological improvement, as it reduces the consumption of computational capacity, battery power, and bandwidth overall across wearable device users overall, as the digital notification will only occur if a user is interested in the product or service of interest rather than for all users of the device, while still achieving the same result (i.e., reaching those individuals most likely to purchase or use the given product or service).

Fraud risk or other risk screening for insurance claims or banking or other activities and situations: Instead of requiring every person (or case) to undergo extensive testing, vetting, background checks, or other requirements for a claim, opening a bank account, cashing a check, etc., the system generates an ensemble test for a particular use case scenario that identifies the small subset of people or cases who actually are an increased risk. This is accomplished by using training data (i.e., a historical dataset of individual cases, with information about various variables and whether or not the particular binary outcome of interest, such as fraud, occurred) to generate an ensemble test (based upon the process described above for how the system generates such custom tests), and then using that generated ensemble test on an individual's data. If that individual is identified as having increased risk under such testing, the bank or insurance agency may require the individual to undergo more extensive vetting and/or risk mitigation. In this manner, the system increases efficiency and reduces costs of the banking institution by reducing the number of testing and procedures required to identify risky individuals. It would also reduce the burden upon the majority of people (or cases) who would need to undergo the extensive testing, vetting, background checks, or other requirements. If the system is automated, such additional testing of risky individuals can be accomplished automatically by the system sending questions to the risky individuals, accessing databases as needed (e.g., across a network or the Internet) to further determine details about the individuals and/or the truthfulness of those individuals), or otherwise confirming details about the individuals.

Colonoscopy screening for colon cancer: Prior to this disclosure, colonoscopies were generally recommended on a periodic basis for all individuals above a certain age, resulting in a large number of false positives, false negatives, and a large amount of testing done on individuals who may have had a low likelihood of colon cancer. Systems configured as disclosed herein can generate an ensemble test to identify the subset of patients who actually are at elevated risk for colon cancer, rather than include the screening of many patients who will not have colon cancer. In this way, the number of patients who need to undergo colonoscopy can be reduced, without reducing the ability to detect colon cancer. For example, instead of screening everyone over a certain age with colonoscopy (thereby leading to a very large number of individuals undergoing unnecessary testing), the system would generate an ensemble test that identifies (e.g., using the testing procedure involving an ensemble of variable combinations disclosed herein) the subset of people who should undergo colonoscopy, without missing positive cases of colon cancer. In some configurations, the results of the system can automatically trigger scheduling, printing and mailing notification letters to patients, emailing patients with appointment information, and/or performing a colonoscopy. In some configurations, performing a medical procedure (such as a colonoscopy) can be done by a human being (e.g., a doctor performs the colonoscopy). In other configurations, the medical procedure can be done by a robot based on the system predictions/results. In such configurations, the robot may be moving automatically, or may be under the direction of medical personnel.

Mammogram screening for breast cancer: Like colonoscopies, systems configured as disclosed herein generates an ensemble test that can identify a subset of patients who at risk for breast cancer rather than include the screening of many patients who will not have breast cancer. In this way, the number of patients who need to undergo a mammogram can be reduced by a large percentage, without reducing the ability to detect breast cancer. In some configurations, the results of the system can automatically trigger scheduling, printing and mailing notification letters to patients, emailing patients with appointment information, and/or performing a mammogram. In some configurations, performing a medical procedure (such as a mammogram) can be done by a human being (e.g., a doctor performs the mammogram). In other configurations, the medical procedure can be done by a robot based on the system predictions/results. In such configurations, the robot may be moving automatically, or may be under the direction of medical personnel.

Other medical issues: Systems configured as disclosed herein can likewise be used to screen for other medical issues, such as but not limited to an individual having a virus or other infection, with the results being used to trigger remedial actions including, but not limited to, providing the individual with medicines, therapies, surgeries, etc., or performing additional testing upon the patient.

Targeting of therapies to individuals with obesity who may be more likely to benefit: For example, the system can generate an ensemble test that can identify a subset of patients with obesity who are at higher risk for heart disease, diabetes, or other health problems. Such individuals can then receive higher priority for expensive and supply-constrained treatments such as weight loss medications or weight loss surgeries. In addition, the system can identify which treatments, if provided to the subset of individuals, are most likely to benefit the subset of individuals. In some configurations, the results of the system can automatically trigger scheduling, dispensing of medications at the pharmacy, mailing of the medications to patients, and/or providing those treatments through a medical device.

Identification of the subset of patients with a disease who truly are at higher risk for hospitalization or other complications: The system can be used to develop ensemble tests (tailored to the particular use case scenarios) that can identify which patients with a given disease are at elevated risk for hospitalization or complications, thereby reducing the number of patients who need to undergo testing, intensive medical care, or treatment. In some configurations, the results of the system can automatically trigger actions identified to begin reducing their risks for hospitalization/complications, such as medical devices that automatically dispense certain medications or modify the dosage of medications already being administered to a patient or laboratory testing machines that automatically perform additional tests upon patient specimens and samples.

Improving supply chain management through better demand forecasting for a particular product or service: The system generates ensemble tests (tailored to the particular use case scenarios) that can predict, based on combinations of variables, when demand will be higher than normal or lower than normal. Inventory can then be adjusted as needed. In some configurations, the system can automatically adjust purchase orders, instruct drones/robots to remove/adjust inventory (e.g., the inventory currently on shelves and/or in stock), apply discounts (e.g., automatically apply a discount to a product and adjust the price within the system; may also cause updated price stickers to be printed for distribution to on-shelf marketing), and/or adjust marketing (e.g., reducing marketing if demand is higher than desired, increasing marketing if demand is lower than desired). Such adjustments in marketing can be made automatically, with the system automatically sending adjustments to an advertising exchange or other system for managing advertisements.

Improved detection of low risk but high impact events: Non-limiting events can include large changes (increase or decrease) in the stock market, the stock market for a particular industry, commodity market, etc. Other such events can include catastrophic weather events (e.g., flooding, damaging storms) or geologic events (e.g., earthquakes, volcanic eruptions). The system can generate ensemble tests (tailored to the particular use case scenarios) that predict when such non-limiting events will occur. In some configurations, the results of the system can automatically trigger real-world adjustments, such as initiating buy/sell transactions on the stock market, adjusting portfolios to account for an improved risk analysis, mechanically opening or closing the gates on a dam to prepare for or address flooding, re-routing electricity through power lines to prepare for or address potential power outages, shutting off machinery or facilities in order to avoid damage or risk of industrial accidents, etc.

Applications based upon real-time or near real-time monitoring of dynamic conditions: The system generates ensemble tests that can be used to monitor dynamically changing states in a real-time or near real-time fashion and then provide an alert or trigger subsequent action based upon detection of a particular signal or other data. For example, a generated test could be connected with health data from wearable devices and rapidly identify when a patient may be at acute risk of a medical complication or other adverse outcome, such as a heart attack, arrhythmia, or fall. Or, as another example, a generated test can rapidly detect when a self-driving car, robot, or other automated or autonomous machine or system may be at risk for an error and then trigger an automatic response, such as shutting down the machine or system, disengaging the autopilot mode, and/or activating an audio alarm or visual alarm to prompt immediate human intervention or monitoring. As another example, a generated test can be combined with artificial intelligence (AI) applications to improve the accuracy of responses to queries and prompts. Such a test could identify (based upon the user's prompt/query or based upon the AI application's response generation process) when there is a risk for hallucination or other error and then prompt corrective measures or at least a pause in the generative AI's response to a query or prompt in order to conduct more extensive accuracy checking. Such a generated test could also reduce the response time or latency for generative AI models. To illustrate, a generated test may obviate the need for generative AI models to routinely pause to assess for accuracy, as the generated test can quickly screen the user prompt/query (or early stages of the AI model's response generation) to evaluate if a pause is actually needed or not.

Once the system has screened cases or samples using an ensemble test (made up of combination tests and/or machine learning model tests), the system can also perform one or more confirmation tests. These confirmation tests can be generated using a subset of the same historical data used to train the screening test, with the result being that the number of false positive results of the screening test are further reduced.

To illustrate, consider the following example: a user uses the system to generate a screening test as described above to identify which customers are likely to click on an advertisement. Instead of a low (e.g., 1 in 1000) clickthrough rate under previously available approaches, the screening/ensemble test generated as disclosed herein can enable identification of a subset of customers who are predicted to be likely to click on the advertisement. Upon targeting this subset of customers, an improved clickthrough rate (e.g., 1 in 50) can be achieved. To further reduce the number of customers who need to be targeted, a second test (a “confirmation test”) can be generated by the system. This confirmation test can be applied to any customer identified using the first test (the screening test) to see if those customers truly are at increased likelihood of clicking on the ad. In this way, the system can identify an even smaller number of customers to target with the ad, and thereby further improving the clickthrough rate beyond the improvements achieved by using the screening test (e.g., a 1 in 20 clickthrough rate).

Continuing with the example of identifying those likely to click on an advertisement, the system first generates a screening test and one or more confirmation tests. The system then executes the screening test upon each potential new customer within a populace and identifies the subset of individuals within that populace who test positive (i.e., those who are predicted as being more likely to click on the ad). Then, the system applies the confirmation test upon any customer who tested positive. For systems configured to execute both the screening test and the confirmation test(s), only those individuals who test positive in both the screening test and the confirmation test will ultimately be identified as the customers to target with the advertisement.

Generation of confirmation tests is executed in a manner similar to that of generating screening tests, as described above (e.g., using one or more combinations of tests to determine if the variables of a given sample meet the identified cutoff points associated with those variables). The difference is that after using the full historical dataset to produce a screening test, the system will only utilize the subset of samples within the historical dataset that the generated screening test would predict are positive. Then, armed with this predicted positive subset of the historical dataset, the system uses the same processes as those involved when the system generates a screening test-identifying the cutoff value for each individual variable, generating and assembling combinations of variables, and constructing an ensemble consisting of multiple combinations, with the resulting ensemble of variable tests becoming the confirmation test.

The generation of confirmation tests can include, in addition to evaluation of the combination tests, evaluation of different machine learning model tests for potential inclusion into the ensemble encompassing the confirmation test. The approach to evaluating various machine learning models and including them in the ensemble for the confirmatory test is similar to that taken for evaluation and inclusion in the ensemble for the screening test that is described earlier. Thus, system-generated confirmatory tests are encompassed by an ensemble consisting of at least one confirmation test and/or one or more machine learning model tests. In this manner, generation of a confirmation test follows the generation of a screening test.

In some configurations, the system can also generate more than one confirmation test. For example, the system can generate a second, third, fourth, or more confirmation tests that are to be used by the system in sequence following application of the first confirmation test upon any new samples predicted by the screening test to be positive. As an example, for a given use case, the system can generate a first, second, and third confirmation test based upon serially filtered historical datasets. Upon being presented with a new sample that the generated screening test predicts to be positive, the generated first confirmation test is applied. If this first confirmation test predicts that this sample is positive, then the second confirmation test is applied. If this second confirmation test predicts that this sample is positive, then the third confirmation test is applied. A sample will only be considered positive overall (and output by the system as such) if a sample is positive at each testing step (i.e., the sample must test positive by the screening test, then the first confirmation test, then the second confirmation test, and then the third confirmation test, etc.). If there is more than one confirmation test, then there will be a change in how each successive confirmation test is generated. For example, a second confirmation test is trained only on data predicted by both the screening test and the first confirmation test to be positive.

To generate the confirmation test (which will be customized to whichever use case scenario is presented to the system), the system first runs the screening test (previously generated based upon the full historical dataset for whichever use case scenario was presented to the system) on the full historical dataset. The system will identify which samples within the full historical dataset are predicted to be positive based upon the screening test.

The system will then use the predicted positive subset to generate the confirmation test. As with the full historical dataset, each sample in the screening-filtered historical dataset will have: (1) a binary value indicating if the sample truly had positive/negative results (e.g., did the person open the advertisement, or not open the advertisement; did the individual actually have cancer, or did they not have cancer); and (2) variables associated with the sample (e.g., in medical examples, the variable data could include an individual's age, gender, age at which family member had cancer, blood pressure, numbers of times per week that red meat is eaten, number of times per day that fruits and vegetables are eaten, minutes of sitting per day, number of times per week that have constipation, number of times per year that have diarrhea, previous medical history, etc.; in advertising examples the variable data could include an individual's age, gender, number of people living in their household, population of the town in which they reside, income, web history, etc.). The variables associated with the sample usually are continuous (i.e., a numerical value) but may be discrete (e.g., binary, such as yes or no, or options contained within a closed set (e.g., A, B, or C)) in some instances. Note that the binary value indicating if the sample truly had positive/negative results is based upon actual outcomes rather than what are the positive/negative results of performing the screening test on the given sample.

In the same manner as disclosed above with respect to the screening test, the system analyzes the screening-filtered historical dataset (i.e., just the predicted positive data) and then generates, for each remaining variable, a Receiver Operating Characteristic (ROC) curve, identifies the cutoff value for each variable, and the ratio of true positive rate (TPR) to false positive rate (FPR), i.e. the TPR/FPR ratio. Note that the ROC curves and TPR/FPR ratio for each variable (in generating the confirmatory test) are not the same as those previously generated for the screening test. This is because the screening-filtered historical dataset will be a subset of the full historical dataset and thus does not have of the exact same set of datapoints. The system then generates all possible different combinations of individual variables, resulting in combination tests (again the system or user can determine the maximum number of variables in any combination test) and/or machine learning model tests, which can be combined to form an ensemble confirmation test having a desired sensitivity and specificity.

Thus, the process to generate a confirmation test is similar to that for generating a screening test. However, the variables that are selected, the cutoff values that are selected for each variable, the selection of which combination of variables are to be included in the various combination tests, and/or the portfolio of combination tests (and/or machine learning models) contained within the ensemble for the confirmation test will not be the same as that for the screening test. This is because the system uses a different training dataset in the generation of the confirmation test than that used to generate the screening test. More specifically, the system uses the full historical dataset in generating the screening test, while the system uses the screening-filtered historical dataset (i.e., the subset of samples within the full historical dataset that are predicted to be positive based upon running the screening test upon the full historical dataset) in generating the confirmation test.

In some configurations, it is possible to generate a second confirmation test that is used after the first confirmation test. In such configurations, as before, the system uses a historical dataset to generate a screening test. Then, using the screening test-filtered historical dataset, the system generates a first confirmation test. At this point the system runs the first confirmation test upon the screening test-filtered historical dataset to identify the subset of samples that the first confirmation test predicts is positive. This identified subset of samples represents a first confirmation test-filtered dataset which is then used by the system as a training dataset to generate a second confirmation test. The screening and confirmation tests can be executed sequentially, such that with each successive test the likelihood of a false positive is reduced. When a negative test result is found no further tests need to be executed (e.g., if the sample fails the screening test, neither a 1^{st} or 2^{nd} confirmation need to be executed; if the sample fails the 1^{st} confirmation test, the 2^{nd} confirmation test does not need to be executed). There is no limit to the number of additional confirmation tests which may be created.

In some configurations, the system can use machine learning models and/or neural network models. Consider the following example. In addition to developing tests associated with individual variables for an end condition (e.g., an individual test identifying where it is more likely than not that a test is positive rather than negative), the system can develop machine learning tests and/or a neural network tests which, relying on the same individual variable and/or additional data, can make a prediction regarding the end condition. Taking the example of breast cancer, the system can therefore generate individual tests for blood-based variables, x-ray analysis, etc., and one or more machine learning tests/neural network tests which can analyze those same inputs (blood-based variables, x-ray analysis, etc.). The machine learning tests/neural network tests can then be treated as individual tests when it comes to forming combination tests and, if desired, ensemble tests. Likewise, machine learning tests and/or neural network tests can be used during confirmation tests or additional screenings in a similar manner as individual variable tests.

Training of the machine learning model tests and/or neural network tests can rely on the historical data (e.g., known outcomes/end results and data associated with individual variables), and can be trained according to any method known to those of skill in the art.

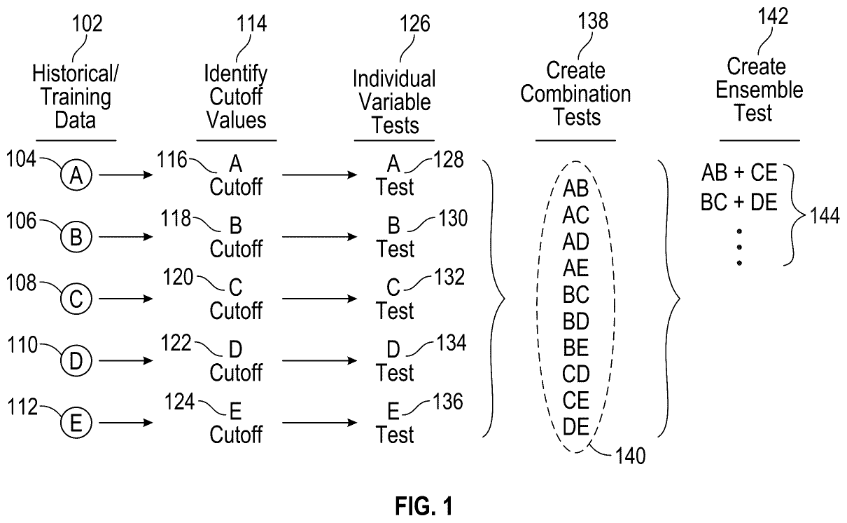

FIG. 1 illustrates an example of the creation of ensemble tests which can be used to filter data. In this example, the system receives historical/training data 102 with variables A 104, B 106, C 108, D 110, and E 112. The system selects which cutoff value is to be used for each individual variable by computing (using the historical/training data) the TPR and FPR for all possible cutoff values for a given variable, thereby generating an ROC curve. The system then identifies which cutoff value will result in the highest TPR/FPR ratio (along with an additional requirement that the TPR is >0.05, or other value if the user would like to specify a different lower limit for the TPR for the cutoff value selection). In this manner, the system selects cutoff values 114 for each of the variables, resulting in an A cutoff 116, B cutoff 118, C cutoff 120, D cutoff 122, and E cutoff 124. Using these cutoff values 116, 118, 120, 122, 124, the system can generate individual variable tests 126, resulting in an A test 128, B test 130, C test 132, D test 134, and E test 136.

Next, the system creates combination tests 138. Preferably, as illustrated, each of the individual tests 128, 130, 132, 134, 136 are combined with all of the other individual tests to form combination tests 140 in the form of pairs (e.g., A test+B test (“AB”), or D test+E test (“DE”)). In some configurations, the system or a user may specify (for computational purposes) the maximum number of variable combinations which may exist in a single combination test. For example, if the user specified a maximum of five optimized individual variable tests per combination, then the combinations to be generated can consist of a pair, triplet, quadruplet, or quintuplet of such tests, but no more than a quintuplet. Likewise, if the user has specified a maximum of three optimized individual variable tests per combination, there will be no more than three variable tests per combination. The number of variables per combination test can vary as needed by a given computational environment, with no upper or lower limits. The system can also create an ensemble test 142, which are a selection of the combination tests 140 (and/or individual tests) to be performed on future samples. For example, the ensemble test may include a combination of the AB combination test and DE combination test, or the AB combination test and the C test 132, etc.

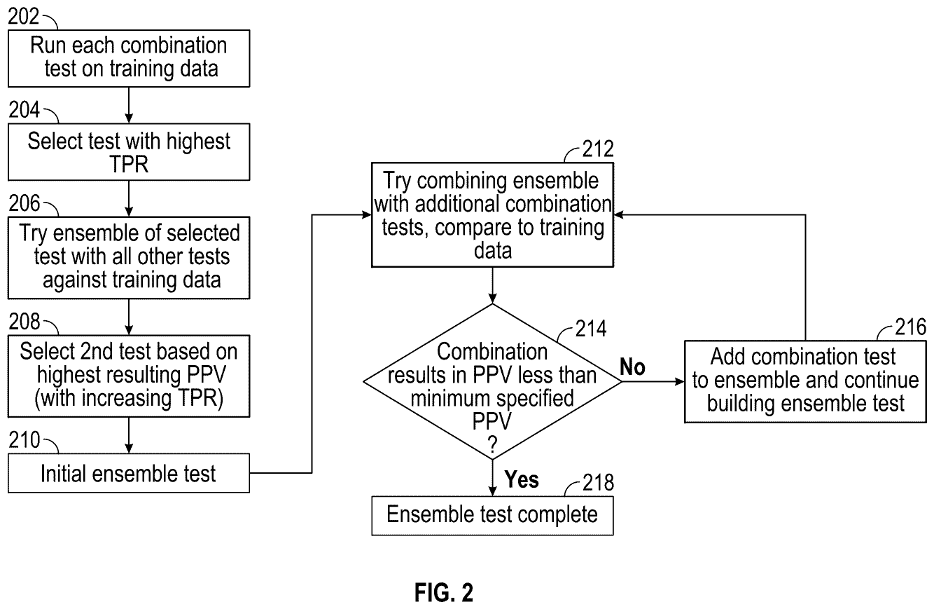

As explained above, and again in the description of FIG. 2, selection of which tests to include in the ensemble test is dependent on the True Positive Rate (TPR) of a proposed test against the historical/training data 102. In other words, once an ensemble test 144 is proposed with a new addition, it can be tested against the historical/training data 102 and, if it fails to meet a desired TPR, the proposed new addition to the ensemble may not be selected. Likewise, if there is a maximum number of tests which can be executed, or other similar restraints on computing power, the tests with the highest TPR will be those selected for future execution.

FIG. 2 illustrates an exemplary process for creating the ensemble test. In this example, combination tests have already been generated, and now the system determines which of those combination tests should be included in the final ensemble of combination tests for use on future samples. First, the system runs each combination test on the training data 202. Here, the system treats each combination test as if it were a prediction test in itself, and then runs each combination test upon all the samples within the training dataset. The system then calculates the True Positive Rate (TPR) for each of the generated combination tests. Next, the system selects the test with the highest TPR to serve as the first combination test to be included in the ensemble. The system then tries an ensemble of the selected test with the other (not yet selected) combination tests against the training data 206, and calculates the TPR and PPV for the proposed ensemble. To run a proposed ensemble test upon a given sample within the training data, the system runs each of the two combination tests (for the particular pair of tests being evaluated) independently upon that sample. If either of the two combination tests is positive, then the overall ensemble test is considered positive (i.e., the ensemble test predicts that the outcome of interest will occur for that particular case represented by the data sample). The system then runs ensemble tests for all samples within the training dataset for the particular proposed ensemble test (i.e., pair or more of combination tests) being evaluated. The system conducts ensemble testing in this manner for every possible pairing of the first combination test in the ensemble with each of the other combination tests available. The system can also calculate the TPR and PPV of these proposed ensemble tests for each possible combination test pair. The system can then select a second combination test to add to the first test based on the highest resulting PPV (with increasing TPR). The first combination test selected 204, and the 2nd combination test selected 208, together form the initial ensemble test 210.

The system can then iteratively probe whether adding further tests (whether combination tests or individual variable tests) to the ensemble would improve the ensemble results. To do so, the system can try combining the ensemble with additional combination tests, then compare the now proposed ensemble to training data 212. Suppose the still-being-assembled ensembled test currently consists of N combination tests, then the system can determine which combination test should be the [N+1th] combination test to include in the growing ensemble. This is done by generating all possible collections of combination tests that can include a possible ensemble test—i.e., all possible collections involving the combination tests already in the ensemble, together with the inclusion of one additional combination test from the remaining pool of available combination tests. For each possible version of the next/proposed, larger ensemble test, the system runs the ensemble testing procedure upon all data in the historical/training data. The system selects the next combination test to be included in the growing ensemble based on which combination test of those available resulted in the overall ensemble test with the highest PPV (that is, the system tests all the possible ensemble tests, then ranks or otherwise identifies which of those had the highest PPV, and selects the combination test which was added to that highest-PPV proposed ensemble test). In addition, inclusion of this additional combination test must also yield an ensemble test with a higher TPR than the TPR from the current version of the ensemble test (i.e., the ensemble without inclusion of the additional combination test).

The system then tests if the proposed combination would result in a PPV less than minimum specified PPV 214. If the answer is no, the system adds the identified combination test to the ensemble test and continues building the ensemble test 216. If the answer is yes, the ensemble test is complete 218, and the system should not add the identified combination test to the ensemble test. At this point the ensemble test is complete, and this is the test that will be used to predict the outcomes from future samples.

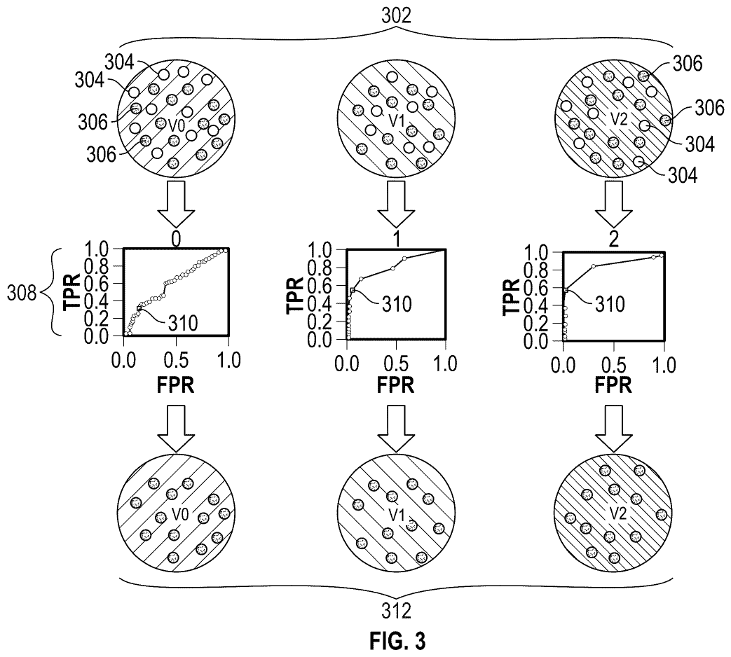

FIG. 3 illustrates an example of three datasets being evaluated using processes disclosed herein. As illustrated, each of the three datasets 302 V0, V1, V2 contain variable data, with white circles 304 illustrating variable data associated with samples which had negative binary results stored in the data for the test/scenario for which the data is being used, and dark circles 306 illustrating variable data associated with samples which had positive binary results for the same test/scenario. As illustrated, the system generates ROC curves 308 for each of the variables based upon the training dataset, the ROC curves 308 plotting the True Positive Rate (TPR) against the False Positive Rate (FPR). The system identifies, for each ROC curve in the ROC curves 308, a TPR/FPR ratio point 310 which corresponds to the highest TPR/FPR ratio (along with an additional requirement that the TPR is >0.05, or other value if the user would like to specify a different lower limit for the TPR for the cutoff value selection). Using this knowledge, the system can determine what cutoff value 310 should be used for that particular variable in defining if a given new sample has a positive or negative result for that variable. The system can then recognize at what point true positive results 312 are likely to be found, resulting in the illustrated (positive only) data sets. Note, the system does not remove the negative (white circle 304) data from the data sets—the illustration of the updated data sets 312 is only to illustrate knowledge of the cutoff point being associated with the positive results (dark circles) 306.

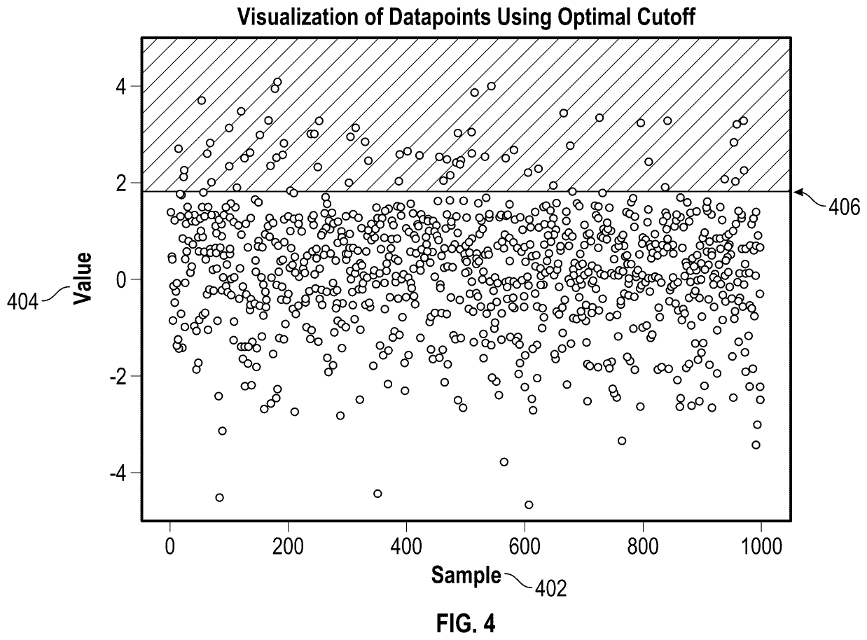

FIG. 4 illustrates an example of a single variable's dataset with a cutoff identified. As described above, the cutoff point for a given variable is identified by the system using the ROC curve for that variable. In this example, any sample with a numerical value higher than the cutoff value is considered to be positive for that variable. In other instances, it is possible to set the cutoff value such that any sample with a numerical value lower than the cutoff value is considered to be positive for that variable.

Importantly, it should be noted that just because an individual variable is positive for a given sample, this does not necessarily mean that the overall test result is considered to have a positive result (i.e., predictive of the binary outcome of interest). The reason why the occurrence of an individual variable being positive should not be sufficient to consider the overall test to be positive is that it otherwise leads to an excessively high rate of false positives. To illustrate, if all it takes for the overall test result to be positive is just for one of the many different variables in a sample to meet the cutoff value, then there will be an excessively high rate of false positives (since use of the selected cutoff value for each individual variable will lead to a certain rate of false positives). Instead, the determination of overall test positivity for a given sample is based upon testing combinations of variables for that sample.

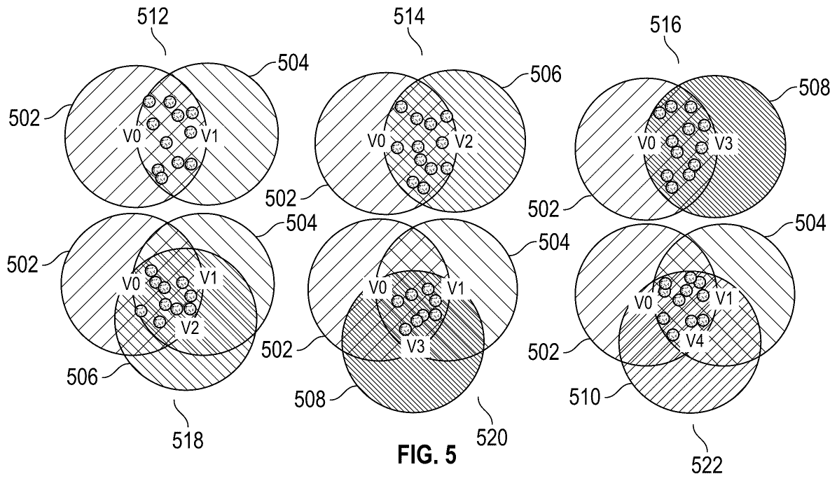

FIG. 5 illustrates an example of combination tests. In this example, pairs of variable tests or triplets of variable tests are combined, resulting in a common test which can test for multiple variables. In this example, there are four different variables: V0 502, V1 504, V2 506, V3 508, and V4 510. The cutoff values for each variable has already been established via the method illustrated in FIG. 3, and further described herein. In FIG. 5, the system is generating possible combination tests which can be tested for selection in the overall ensemble test. As illustrated, a first exemplary combination test 512 can be of V0 502 and V1 504. A second exemplary combination test 514 can be of V0 502 and V2 506. A third exemplary combination test 516 can be of V0 502 and V3 508. The combination tests do not need to be just pairs of individual variable tests, but rather can be combinations of any number of individual variable tests. A fourth exemplary combination test 518 can be of V0 502, V1 504, and V2 506. A fifth exemplary combination test 520 can be of V0 502, V1 504, and V3 508. A six exemplary combination test 522 can be of V0 502, V1 504, and V4 510.

For a given combination, the combination (or combination test) will be considered positive (i.e., predictive that the binary outcome of interest will occur) for a given test sample only if every variable within that combination is positive (i.e., the sample's numerical value for that variable exceeds the specified cutoff value). This requirement for all variables within a combination needing to be positive in order for the combination to be positive is illustrated in this figure (FIG. 5). Each circle represents the collection of samples that are positive for a particular variable. Only the subset of samples residing within the overlapping portion of circles (i.e., the overlap represents samples that have positive values for each of the various variables) are considered to have a positive combination of variables.

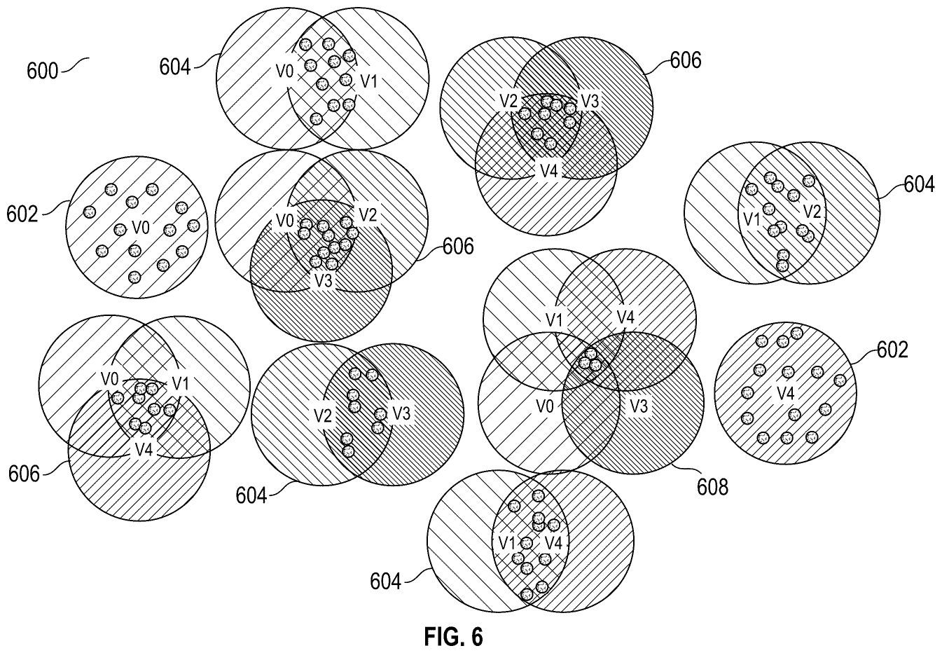

FIG. 6 illustrates an example of an ensemble test 600, which consists of multiple different combinations tests (each consisting of a single or multiple variables). In this example, the ensemble includes combination tests consisting of a single variable 602 as well as multi-variable combination tests (pairs 604, triplets 606, or quadruplets 608). When the ensemble test 600 is executed (i.e., the selected tests are run on new sample data) on a given test sample, the sample's numerical value for each variable is evaluated to see if it exceeds the cutoff value for that variable and thereby if the sample is positive for that variable. Then, the testing procedure involves evaluating each of the combination tests 602, 604, 606, 608 on the sample to determine if the combination tests 602, 604, 606, 608 result in positive outcomes. As described before, in order for a given combination test to be positive, every individual variable test in that combination test must be positive. As long as at least one of the combination tests 602, 604, 606, 608 within the ensemble test 600 is positive, then the overall test result for that sample will be labeled positive (i.e., predictive of the binary outcome of interest).

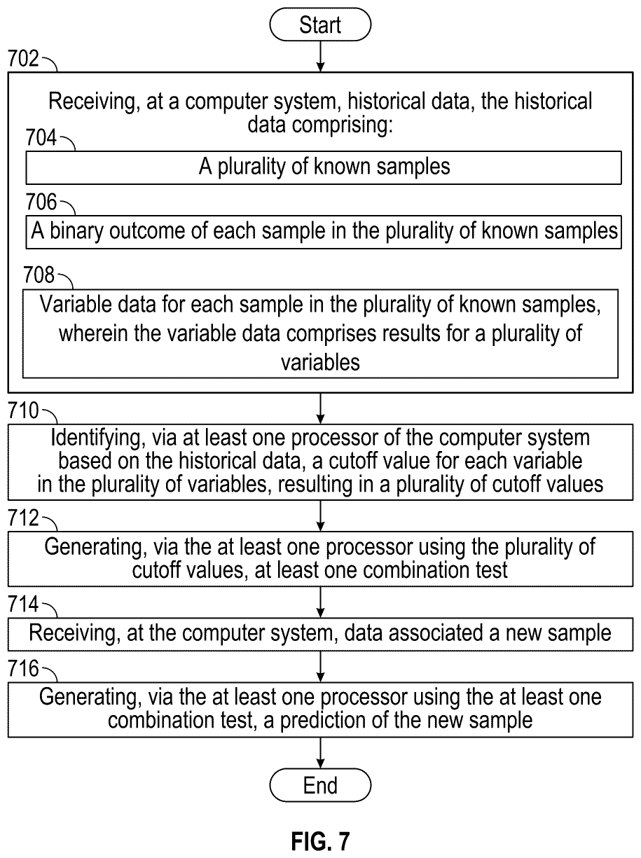

FIG. 7 illustrates an example method embodiment. As illustrated, the method can include receiving, at a computer system, historical data (702), the historical data comprising: a plurality of known samples (704); a binary outcome (e.g., positive or negative) of each sample in the plurality of known samples (706); and variable data for each sample in the plurality of known samples, wherein the variable data comprises results for a plurality of variables (708). The method can next include identifying, via at least one processor of the computer system based on the historical data, a cutoff value for each variable in the plurality of variables (e.g., using ROC curves as described above), resulting in a plurality of cutoff values (710), and generating, via the at least one processor using the plurality of cutoff values, at least one combination test (712). The illustrated method can conclude with receiving, at the computer system, data associated a new sample (714) and generating, via the at least one processor using the at least one combination test, a prediction of the new sample (716).

In some configurations, the identifying of the cutoff value for each variable can further include: generating a Receiver Operating Characteristic (ROC) curve for each variable in the plurality of variables, the ROC curve plotting the binary outcome against the each variable, resulting in a plurality of ROC curves where each ROC curve in the plurality of ROC curves is associated with a variable in the plurality of variables; identifying a TPR/FPR ratio point corresponding to the highest TPR/FPR ratio (along with an additional requirement that the TPR is >0.05 (or other value if the user would like to specify a different lower limit for the TPR for the cutoff value selection)) within each ROC curve in the plurality of ROC curves. The value of the variable at the highest TPR/FPR ratio point being associated with a value for the variable in the plurality of variables, wherein the value is the cutoff value. For example, the system uses the ROC curve, identifying for each value of the variable the TPR and FPR. The system detects that the highest ratio found is a ratio of 4 (i.e., TPR/FPR is 4), with the corresponding TPR being 20% and FPR 5%. The corresponding value of the variable at that point is (in this example) 224 (out of a possible 1000), such that the cutoff value is 224. Thus, the cutoff value is not the same numerical value as the TPR/FPR ratio, but corresponds to the value of the variable at that point of highest ratio.

In some configurations, the generating of the at least one combination test can include: for each variable in the plurality of variables: combining the cutoff value for the each variable with at least one cutoff value for at least additional variable from the plurality of variables, resulting in combined cutoff values, wherein the combined cutoff values result in a combination test where each cutoff value in the combined cutoff values must be met for a positive result. In such configurations, the combined cutoff values can result in combinations of at least three variables within the plurality of variables.

In some configurations, the illustrated method can further include: for each combination test in the at least one combination test, calculating a True Positive Rate (TPR) using the combination test on the historical data, resulting in TPR results; identifying a highest scoring combination test within the at least one combination test based on the TPR results; selecting the highest scoring combination test as one of at least one combination test to be performed on the new sample, wherein the generating of the prediction of the new sample uses the at least one combination to be performed. In such configurations, the selecting of the highest scoring combination test as one of the at least one combination test to be performed can continue until a predetermined maximum number of combination tests to be performed is reached.

In some configurations, if any result of the at least one combination test provides a positive result regarding the new sample, the new sample tests positive for a given condition.

In another configuration of the system, the system can also include various machine learning models (as well as combination tests) in the ensemble test. The system will use the particular historical dataset for the given use case as a training dataset. Just as how it was described earlier and depicted in FIG. 1, the system will generate a pool of combination tests that are now candidates for potential inclusion in the ensemble test. This pool of candidates for potential inclusion in the ensemble test will be broadened by also adding in various machine learning models. To do so, the system first runs each machine learning model on the historical dataset for the particular use case. Using this historical dataset, the system will calculate the PPV for each of the machine learning models. This PPV is calculated for each machine learning model by treating each machine learning model as if it were a standalone test and then evaluating that machine learning model's ability to correctly predict true positives by surveying every sample within the historical dataset. Then, the system will identify and select only the subset of machine learning models that yield a PPV that is at least as high as a minPPV that is specified by the user.

If a given machine learning model (whether it is the default version or a variant of it) offers a PPV that is at least as high as the user-specified minPPV, then this machine learning model is included in the pool of candidates for potential inclusion in the in the ensemble.

Next, the system will assemble together the ensemble test (i.e., the collection of combination tests and machine learning models) to be used. The system will first determine the TPR for each combination test as well as for each machine learning model that has been identified as a candidate for potential inclusion in the ensemble. The system does this by treating each combination test as if it were a prediction test in itself and then runs each combination test upon all the samples within the training dataset (i.e., historical dataset). Based upon these runs, the system calculates the True Positive Rate (TPR) for each of the generated combination tests. In addition, the system will also calculate the TPR for each machine learning model that had been identified as having a PPV at least as high as the user-specific minPPV. This calculation is conducted by running the given machine learning model upon all samples within the training dataset.

Among the pool of candidate ensemble members (whether a combination test or machine learning model), the system selects the one with the highest TPR to serve as the first member to be included in the ensemble. The system then generates every possible pairing of this first member with all the other possible remaining candidate members (whether combination test or machine learning model). Each of these possible pairs (which could be a pair of combination tests, a pairing of a combination test with a machine learning model, or a pair of machine learning models) are then evaluated as if they were actual final ensemble test. In such a test, the system runs each of the two proposed ensemble members independently upon that sample. If either of the two ensemble members is positive, then the overall ensemble test is considered positive (i.e., the ensemble test predicts that the outcome of interest will occur for that particular case represented by the data sample).

In this manner, the system applies this testing for each of the possible ensembles upon each of the samples within the training data. Based upon the results, the system calculates the TPR and PPV for every possible ensemble. The second member that, upon pairing with the first already selected member, yields an ensemble test with the highest resulting PPV (while still increasing the TPR higher than that for an ensemble consisting of only the first member) is then selected for including in the growing ensemble.

The system can then grow the ensemble further, with the addition of one new member per growth cycle. Suppose the still-being-assembled ensembled test currently consists of N members (which could be a mix of combination tests and/or machine learning models), then the system can determine which candidate ensemble member should be the [N+1th] member to include in the growing ensemble. This is done by generating all possible collections of members that can be included in a possible ensemble test—i.e., all possible collections involving the members already in the ensemble, together with the inclusion of one additional member from the remaining pool of available candidates (i.e., combination tests and machine learning models). For each possible version of the next proposed, larger ensemble test, the system runs the ensemble testing procedure upon all samples in the historical/training data. The system selects the next member to be included in the growing ensemble based on which member of those still available will lead to the overall ensemble test having the highest PPV (that is, the system tests all the possible ensemble tests, then ranks or otherwise identifies which of those had the highest PPV, and then selects the member which had been added to that highest-PPV proposed ensemble test). In addition, inclusion of this additional member must also yield an ensemble test with a higher TPR than the TPR from the current version of the ensemble test (i.e., the ensemble without inclusion of the additional member).

The system then tests if the proposed ensemble would result in an ensemble test PPV that is less than minimum specified PPV. If the answer is no, the system adds the identified member to the ensemble test and continues building the ensemble test. If the answer is yes, the ensemble test is complete, and the system should not add the identified member to the ensemble test. At this point the ensemble test is complete, and this is the test that will be used to predict the outcomes from future samples.

The size of the overall ensemble test will vary depending upon the particular use case scenario (and its associated training dataset) and the user-specified minPPV that the user is employing. For example, a given ensemble test could include 1, 2, 3, 4, 5, or more than 5 members (consisting of different combination tests, and may also include different machine learning models). Thus, system-generated tests are encompassed by an ensemble consisting of at least one confirmation test and which may also include one or more machine learning models.

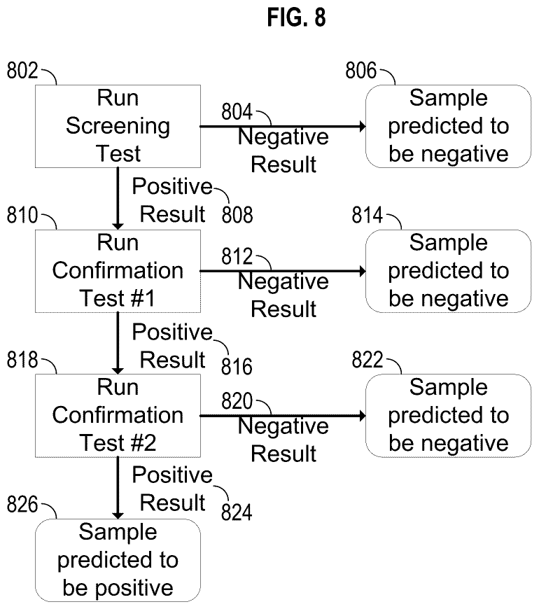

FIG. 8 illustrates an example of multiple confirmation tests 810, 818 following a screening test 802. In this particular example, the system has generated two confirmatory tests 810, 818 that can be used upon any sample predicted as positive by a system-generated screening test 802.

Upon receipt of a new sample, the system first applies the screening test 802. If the screening test 802 predicts a negative result 804, predicting that the new sample is negative 806, then no further testing is done. If the screening test 802 predicts a positive result 808, then the system executes the first confirmation test 810. If the first confirmation test 810 predicts a negative result 812, predicting that the new sample is negative 814, then no further testing is done. If the first confirmation test 810 predicts a positive result 816, then the system executes the second confirmation test 818. If the second confirmation test 818 predicts a negative result 820, predicting that the new sample is negative 822, then no further testing is done. If the second confirmation test 818 predicts a positive result 826, then the system outputs an overall prediction that the sample is positive.

Thus, a sample will only be predicted as positive if the sample is positive at each testing step upon sequential testing with the screening test, first confirmatory test, and second confirmatory test.

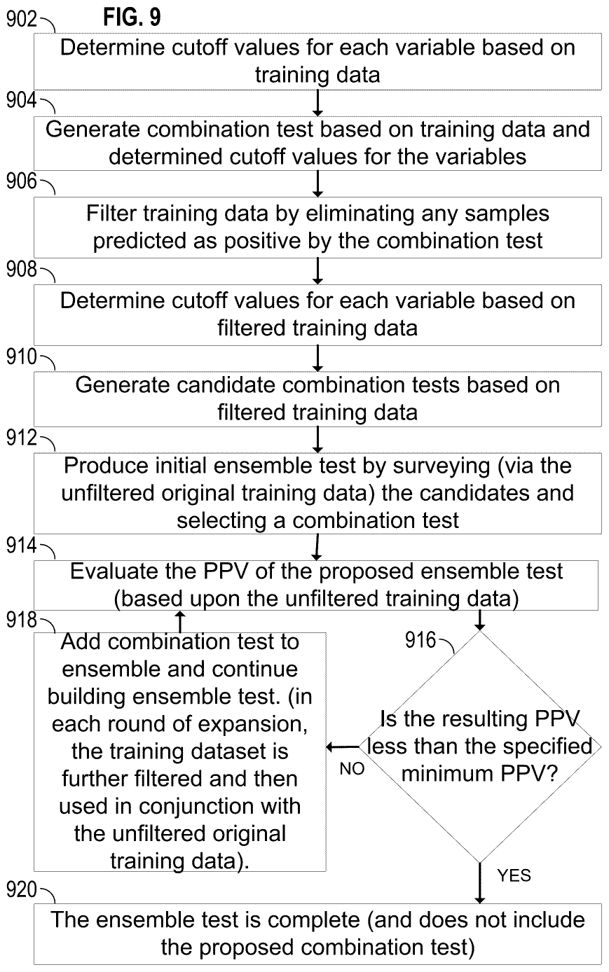

FIG. 9 illustrates an exemplary process for creating ensemble tests based upon recursive filtering of the training data. In this example, cutoff values are determined for each variable based upon the historical dataset (902). Candidate combinations of the various variables are then generated (904), and then the system determines which of those combination tests should be included in the final ensemble of combination tests for use on future samples. To do so, the system runs each combination test on the training data. Here, the system treats each combination test as if it were a prediction test in itself and then runs each combination test upon all the samples within the training dataset. The system then calculates the Positive Predictive Value (PPV) for each of the generated combination tests and selects the test with the highest PPV to serve as the first combination test to be included in the ensemble.

The system then filters the historical dataset by removing any samples predicted positive by this combination test (906). Using this filtered historical dataset, the system determines the cutoff values for each variable (908). The system then generates candidate combinations of the various variables based on the filtered training data (910). To be eligible as a candidate combination for consideration, the TPR for a given combination must be at least as high as a minimum value that the user specifies. (It should be noted that this user-specified value for candidate combinations is distinct from the user-specified minimum TPR for the overall test that in turn is used to calculate the minPPV). Calculation of the TPR for each candidate combination is accomplished by trying this combination test upon every sample in the filtered, historical dataset.

Next, the system decides which of those combination tests should be included in the final ensemble of combination tests for use on future samples. The system tries an ensemble of the first selected combination test paired with the other (not yet selected) combination tests against the full, original training dataset (i.e., the unfiltered historical dataset) and calculates the PPV for the proposed ensemble, evaluating the PPV of the proposed ensemble test (based upon the unfiltered training data) (914). To run a proposed ensemble test upon a given sample within the training data, the system runs each of the two combination tests (for the particular pair of tests being evaluated) independently upon that sample. If either of the two combination tests is positive, then the overall ensemble test is considered positive (i.e., the ensemble test predicts that the outcome of interest will occur for that particular case represented by the data sample). The system then runs ensemble tests for all samples within the training dataset for the particular proposed ensemble test (i.e., pair or more of combination tests) being evaluated. The system conducts ensemble testing in this manner for every possible pairing of the first combination test in the ensemble with each of the other combination tests available. The system then selects a second combination test to add to the first test based on the highest resulting PPV (with increasing TPR).

The system can then iteratively probe whether adding further combination tests to the ensemble would improve the ensemble results. Each round of expansion proceeds in a similar fashion as the prior round. The prior round's training dataset is filtered by excluding samples predicted as positive by the ensemble generated from that prior training dataset. A cutoff value for each variable is determined based upon this iteration of the training dataset. The system generates various candidate combination tests using these determined cutoff values for each variable. Candidate combinations must have a TPR (calculated using the current iteration of the filtered training dataset) greater than the user-specified minimum value. Again, a combination test is positive if and only if every variable in the combination is positive for the sample.

The system selects from, amongst the pool of generated candidate combination tests, a combination test to add to the prior ensemble. This selection decision is based upon identifying which combination test, upon being added to the ensemble, will lead to the ensemble test having the highest PPV (which is calculated based upon running the proposed ensemble test upon the original training dataset, i.e. the unfiltered historical dataset). In addition, inclusion of this additional combination test must also yield an ensemble test with a higher TPR than the TPR from the current version of the ensemble test (i.e., the ensemble without inclusion of the additional combination test). This TPR calculation is based upon the original training dataset.

The system then tests if the proposed ensemble including this additional combination test would result in a PPV less than minimum specified PPV (916). If the answer is no, the system adds the identified combination test to the ensemble test and continues building the ensemble test (918). Further rounds of ensemble construction will be conducted as before (including the iterative filtering process for the training dataset). If, however, the answer is yes, then the ensemble test is complete (920), and the system should not add the identified combination test to the ensemble test. At this point the ensemble test is complete, and this is the test that will be used to predict the outcomes from future samples.

For this version of the system involving the use of recursive filtering of the training dataset, machine learning model tests, as well as combination tests, can also be included in the ensemble. Thus, generated tests could therefore be an ensemble consisting of at least one combination test, together with additional combination test(s) and/or machine learning model(s).

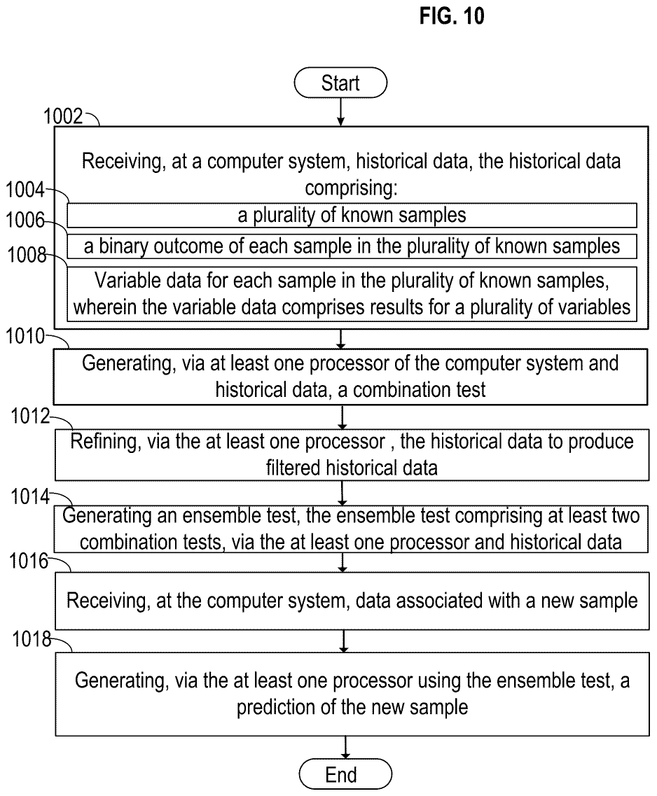

FIG. 10 illustrates an example method embodiment. As illustrated, the method can include receiving, at a computer system, historical data (1002), the historical data comprising: a plurality of known samples (1004); a binary outcome (e.g., positive or negative) of each sample in the plurality of known samples (1006); and variable data for each sample in the plurality of known samples, wherein the variable data comprises results for a plurality of variables (1008). The method can next include generating, via at least one processor of the computer system and historical data, a combination test (1010). This can include identifying, via at least one processor of the computer system and using the historical data, a cutoff value for each variable in the plurality of variables (e.g., using ROC curves as described above), resulting in a plurality of cutoff values, and then generating the at least one combination test using the plurality of cutoff values. The method continues by refining the historical data to produce filtered historical data (1012). This can include removing samples already predicted as positive by the first combination test. An ensemble (of at least two combination tests) is then generated (1014), via the at least one processor of the computer system, by using the filtered and unfiltered versions of the historical data to generate (and add to the ensemble) a new combination test. Via the at least one processor of the computer system, the ensemble test can continue to grow to completion through use of the historical dataset (in both the unfiltered and iteratively filtered forms). The illustrated method can conclude with receiving, at the computer system, data associated with a new sample (1016) and generating, via the at least one processor of the computer system and using the ensemble test, a prediction of the new sample (1018).

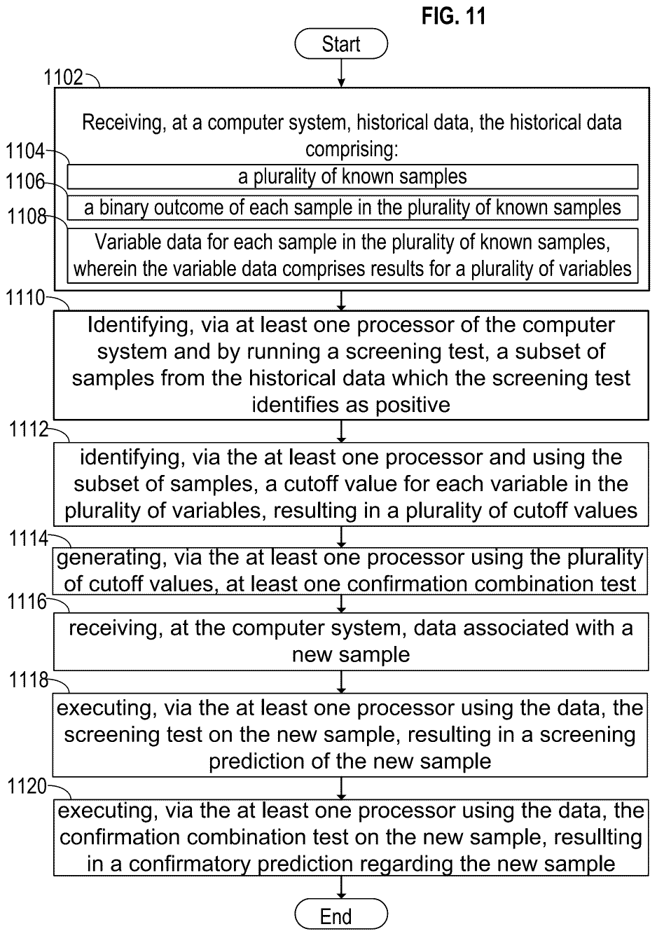

FIG. 11 illustrates an example confirmation method embodiment. As illustrated, the method can include receiving, at a computer system, historical data (1102), the historical data comprising: a plurality of known samples (1104), a binary outcome of each sample in the plurality of known samples (1106), and variable data for each sample in the plurality of known samples, wherein the variable data comprises results for a plurality of variables (1108). Next, the method can include identifying, via at least one processor of the computer system and by running a screening test, a subset of samples from the historical data which the screening test identifies as positive (1110), and identifying, via the at least one processor and using the subset of samples, a cutoff value for each variable in the plurality of variables, resulting in a plurality of cutoff values (1112). The method can next include generating, via the at least one processor using the plurality of cutoff values, at least one confirmation combination test (1114), and receiving, at the computer system, data associated with a new sample (1116). The method can then include executing, via the at least one processor using the data, the screening test on the new sample, resulting in a screening prediction of the new sample (1118) and executing, via the at least one processor using the data, the confirmation combination test on the new sample, resulting in a confirmation prediction regarding the new sample (1120).

In another configuration of the system, the system can also include various machine learning models (as well as combination tests) in the ensemble test for use as a confirmation test. Confirmation tests could therefore be an ensemble consisting of at least one combination test, together with additional combination test(s) and/or machine learning model(s).

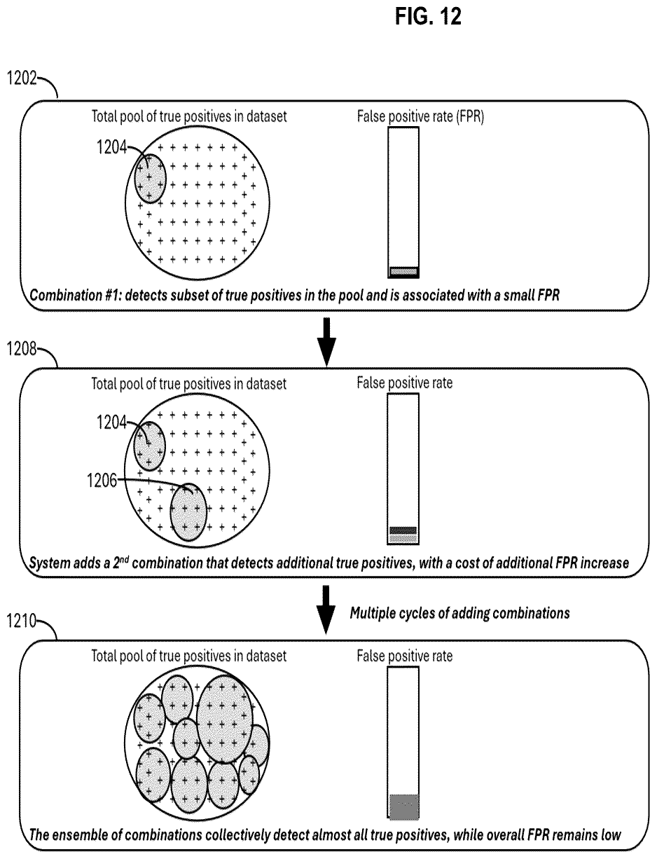

FIG. 12 illustrates an example of the impact of a recursive dataset filtering approach in constructing an ensemble. By filtering out historical data samples already predicted as positive by the current version of the still growing ensemble, then the next combination test can be generated from the filtered historical data towards an aim of capturing true positives not already captured, while minimizing the increase to the overall false positive rate of the ensemble. In the first step 1202, a single combination test associated with a first portion 1204 of the total pool of true positives is selected to be part of an ensemble test. Selection of the first combination test can be made according to the concepts and principles disclosed herein. While the combination test is selected based on the ability to predict a “true positive”, false positives will, in most cases, exist—the systems and methods disclosed herein only seek to minimize those false positives. Thus, selection of the first combination test in step 1202 increases the false positive rate (FPR) slightly.

The ensemble test selection process continues in step 1204, where a second combination test is selected, the second combination test covering a distinct portion 1206 of the total pool of true positives than the first portion 1204 of the total pool of true positives in the dataset. The recursive dataset filtering approach serves to minimize or eliminate the overlap in the subset of true positives detected by the different combination tests. As illustrated, there is no overlap between the first portion 1204 (covered by the first selected combination test) and the second, distinct portion 1206 (covered by the second selected combination test). Again, the FPR increases slightly with the added test.

This process of adding additional tests which have low or no overlap with other tests in the total pool of true positives in the dataset can continue until there are no remaining tests available which would (according to the principles disclosed herein) improve the overall test results of the ensemble tests. As illustrated in the final step 1210, all portions corresponding to individual combination tests selected for the ensemble test have low or no overlap with other portions, while the overall false positive rate of the ensemble test remains low.

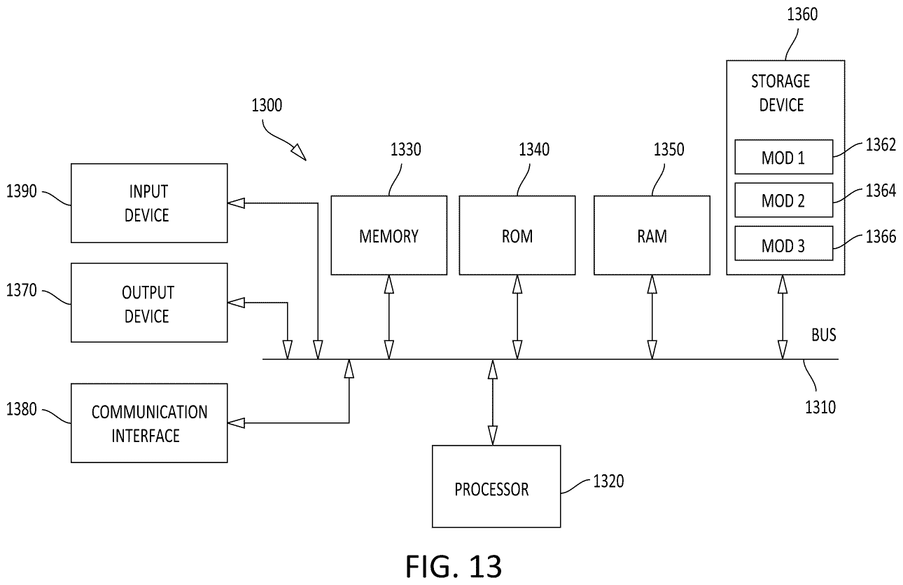

With reference to FIG. 13, an exemplary system includes a computing device 1300 (such as a general-purpose computing device), including a processing unit (CPU or processor) 1320 and a system bus 1310 that couples various system components including the system memory 1330 such as read-only memory (ROM) 1340 and random access memory (RAM) 1350 to the processor 1320. The computing device 1300 can include a cache of high-speed memory connected directly with, in close proximity to, or integrated as part of the processor 1320. The computing device 1300 copies data from the system memory 1330 and/or the storage device 1360 to the cache for quick access by the processor 1320. In this way, the cache provides a performance boost that avoids processor 1320 delays while waiting for data. These and other modules can control or be configured to control the processor 1320 to perform various actions. Other system memory 1330 may be available for use as well. The system memory 1330 can include multiple different types of memory with different performance characteristics. It can be appreciated that the disclosure may operate on a computing device 1300 with more than one processor 1320 or on a group or cluster of computing devices networked together to provide greater processing capability. The processor 1320 can include any general-purpose processor and a hardware module or software module, such as module 1 1362, module 2 1364, and module 3 1366 stored in storage device 1360, configured to control the processor 1320 as well as a special-purpose processor where software instructions are incorporated into the actual processor design. The processor 1320 may essentially be a completely self-contained computing system, containing multiple cores or processors, a bus, memory controller, cache, etc. A multi-core processor may be symmetric or asymmetric.

The system bus 1310 may be any of several types of bus structures including a memory bus or memory controller, a peripheral bus, and a local bus using any of a variety of bus architectures. A basic input/output (BIOS) stored in memory ROM 1340 or the like, may provide the basic routine that helps to transfer information between elements within the computing device 1300, such as during start-up. The computing device 1300 further includes storage devices 1360 such as a hard disk drive, a magnetic disk drive, an optical disk drive, tape drive or the like. The storage device 1360 can include software modules 1362, 1364, 1366 for controlling the processor 1320. Other hardware or software modules are contemplated. The storage device 1360 is connected to the system bus 1310 by a drive interface. The drives and the associated computer-readable storage media provide nonvolatile storage of computer-readable instructions, data structures, program modules and other data for the computing device 1300. In one aspect, a hardware module that performs a particular function includes the software component stored in a tangible computer-readable storage medium in connection with the necessary hardware components, such as the processor 1320, system bus 1310, output device 1370 (such as a display or speaker), and so forth, to carry out the function. In another aspect, the system can use a processor and computer-readable storage medium to store instructions which, when executed by a processor (e.g., one or more processors), cause the processor to perform a method or other specific actions. The basic components and appropriate variations are contemplated depending on the type of device, such as whether the computing device 1300 is a small, handheld computing device, a desktop computer, or a computer server.

Although the exemplary embodiment described herein employs the storage device 1360 (such as a hard disk), other types of computer-readable media which can store data that are accessible by a computer, such as magnetic cassettes, flash memory cards, digital versatile disks, cartridges, random access memories (RAMs) 1350, and read-only memory (ROM) 1340, may also be used in the exemplary operating environment. Tangible computer-readable storage media, computer-readable storage devices, or computer-readable memory devices, expressly exclude media such as transitory waves, energy, carrier signals, electromagnetic waves, and signals per se.

To enable user interaction with the computing device 1300, an input device 1390 represents any number of input mechanisms, such as a microphone for speech, a touch-sensitive screen for gesture or graphical input, keyboard, mouse, motion input, speech and so forth. An output device 1370 can also be one or more of a number of output mechanisms known to those of skill in the art. In some instances, multimodal systems enable a user to provide multiple types of input to communicate with the computing device 1300. The communications interface 1380 generally governs and manages the user input and system output. There is no restriction on operating on any particular hardware arrangement and therefore the basic features here may easily be substituted for improved hardware or firmware arrangements as they are developed.

The technology discussed herein refers to computer-based systems and actions taken by, and information sent to and from, computer-based systems. One of ordinary skill in the art will recognize that the inherent flexibility of computer-based systems allows for a great variety of possible configurations, combinations, and divisions of tasks and functionality between and among components. For instance, processes discussed herein can be implemented using a single computing device or multiple computing devices working in combination. Databases, memory, instructions, and applications can be implemented on a single system or distributed across multiple systems. Distributed components can operate sequentially or in parallel.

Neural networks, foundational to modern artificial intelligence, are computational systems designed to process data and generate predictions or classifications by emulating aspects of human brain function. A neural network is a framework of machine learning algorithms that work together to classify inputs based on a previous training process. They power applications like image recognition, natural language processing, and predictive analytics. At their core, neural networks consist of interconnected layers of mathematical units called neurons, organized into an input layer, one or more hidden layers, and an output layer. The input layer receives raw or preprocessed data, such as pixel values or text embeddings, represented as numerical vectors. Hidden layers transform this data into increasingly abstract representations through complex computations, while the output layer produces the result, such as a class probability or a numerical prediction. Each neuron connects to those in the next layer via weighted connections, where weights are numerical values that amplify or diminish the influence of one neuron's output on another's input. Additionally, biases—adjustable offsets—enhance the model's flexibility in fitting data.

The operation of a neural network begins with a forward pass, where data flows from the input layer through the hidden layers to the output. Each neuron computes a weighted sum of its inputs, adds its bias, and applies a nonlinear activation function, such as a sigmoid, rectified linear unit (ReLU), or hyperbolic tangent (tanh), to produce an output. This process repeats across layers, with each layer extracting more complex features, such as edges in images or semantic patterns in text. The final layer's output depends on the task: classification tasks yield probabilities (e.g., “90%”), while regression tasks produce continuous values (e.g., a predicted temperature). Crucially, the forward pass does not alter the model's stored parameters—weights and biases—which represent the network's learned knowledge. These parameters are stored in digital memory, typically as 32-bit or 16-bit floating-point arrays. Weights form matrices, with rows and columns corresponding to neurons in adjacent layers, while biases are stored as one-dimensional arrays. Meta-information, such as layer counts and activation function types, is also stored to define the network's structure.

Training a neural network involves adjusting its parameters to minimize prediction errors. During training, a forward pass generates predictions, which are compared to correct outputs using a loss function, such as mean squared error or cross-entropy, to quantify errors. Backpropagation then computes gradients, indicating how much each parameter contributed to the error, by applying the chain rule to propagate errors backward from the output to the input layer. Optimization algorithms, like stochastic gradient descent, adjust weights and biases in directions that reduce the loss. This process iterates over multiple epochs, with parameters gradually converging to values that improve accuracy. Memory usage during training is dynamic: weights and biases are updated incrementally for each data batch, and intermediate results, like neuron activations and gradients, are temporarily stored in buffers to facilitate backpropagation. To ensure progress is saved, parameters are periodically checkpointed to persistent storage, allowing training to resume later. Efficiency techniques, such as reducing parameter precision to 16-bit formats, further optimize memory and computation.

Once trained, the network enters inference mode, where parameters are fixed, and only forward passes are executed to generate predictions. This mode minimizes memory writes, making it ideal for deployment on resource-constrained devices like mobile phones. From a patent perspective, innovations in neural networks often focus on novel storage architectures to reduce memory usage, unique parameter update mechanisms to enhance training efficiency, hybrid memory systems combining volatile and non-volatile storage, or dynamic precision adjustments during training or inference.

Use of language such as “at least one of X, Y, and Z,” “at least one of X, Y, or Z,” “at least one or more of X, Y, and Z,” “at least one or more of X, Y, or Z,” “at least one or more of X, Y, and/or Z,” or “at least one of X, Y, and/or Z,” are intended to be inclusive of both a single item (e.g., just X, or just Y, or just Z) and multiple items (e.g., {X and Y}, {X and Z}, {Y and Z}, or {X, Y, and Z}). The phrase “at least one of” and similar phrases are not intended to convey a requirement that each possible item must be present, although each possible item may be present.

The various embodiments described above are provided by way of illustration only and should not be construed to limit the scope of the disclosure. Various modifications and changes may be made to the principles described herein without following the example embodiments and applications illustrated and described herein, and without departing from the spirit and scope of the disclosure. For example, unless otherwise explicitly indicated, the steps of a process or method may be performed in an order other than the example embodiments discussed above. Likewise, unless otherwise indicated, various components may be omitted, substituted, or arranged in a configuration other than the example embodiments discussed above.

Further aspects of the present disclosure are provided by the subject matter of the following clauses.

A method, comprising: receiving, at a computer system, historical data, the historical data comprising: a plurality of known samples; a binary outcome of each sample in the plurality of known samples; and variable data for each sample in the plurality of known samples, wherein the variable data comprises results for a plurality of variables; identifying, via at least one processor of the computer system based on the historical data, a cutoff value for each variable in the plurality of variables, resulting in a plurality of cutoff values; generating, via the at least one processor using the plurality of cutoff values, at least one combination test; receiving, at the computer system, data associated a new sample; and generating, via the at least one processor using the at least one combination test, a prediction of the new sample.

A method comprising: receiving, at a computer system, historical data, the historical data comprising: a plurality of known samples; a binary outcome of each sample in the plurality of known samples; variable data for each sample in the plurality of known samples, wherein the variable data comprises results for a plurality of variables; identifying, via at least one processor of the computer system and by running a screening test, a subset of samples from the historical data which the screening test identifies as positive; identifying, via the at least one processor and using the subset of samples, a cutoff value for each variable in the plurality of variables, resulting in a plurality of cutoff values; generating, via the at least one processor using the plurality of cutoff values, at least one confirmation combination test; receiving, at the computer system, data associated with a new sample; executing, via the at least one processor using the data, the screening test on the new sample, resulting in a screening prediction of the new sample; and executing, via the at least one processor using the data, the confirmation combination test on the new sample, resulting in a confirmation prediction regarding the new sample.

A method, comprising: receiving, at a computer system, historical data, the historical data comprising: a plurality of known samples; a binary outcome of each sample in the plurality of known samples; and variable data for each sample in the plurality of known samples, wherein the variable data comprises results for a plurality of variables; identifying, via at least one processor of the computer system based on the historical data, a cutoff value for each variable in the plurality of variables, resulting in a plurality of cutoff values; generating, via the at least one processor using the plurality of cutoff values, at least one combination test; generating, via the at least one processor using at least one machine learning model and the historical data, at least one machine learning model test; receiving, at the computer system, data associated a new sample; and generating, via the at least one processor using at least one of the at least one combination test or the at least one machine learning model test, a prediction of the new sample.

A method comprising: receiving, at a computer system, historical data, the historical data comprising: a plurality of known samples; a binary outcome of each sample in the plurality of known samples; variable data for each sample in the plurality of known samples, wherein the variable data comprises results for a plurality of variables; training a neural network using the historical data; identifying, via at least one processor of the computer system and by running a screening test, a subset of samples from the historical data which the screening test identifies as positive; identifying, via the at least one processor and using the subset of samples, a cutoff value for each variable in the plurality of variables, resulting in a plurality of cutoff values; generating, via the at least one processor using the plurality of cutoff values and the neural network, at least one confirmation combination test; receiving, at the computer system, new sample data associated with a new sample; executing, via the at least one processor using the data, the screening test on the new sample data, resulting in a screening prediction of the new sample; and executing, via the at least one processor using the data, the confirmation combination test on the new sample data, resulting in a confirmation prediction regarding the new sample.

A method, comprising: receiving, at a computer system, historical data, the historical data comprising: a plurality of known samples; a binary outcome of each sample in the plurality of known samples; and variable data for each sample in the plurality of known samples, wherein the variable data comprises results for a plurality of variables; training at least one machine learning model using the historical data; identifying, via at least one processor of the computer system based on the historical data, a cutoff value for each variable in the plurality of variables, resulting in a plurality of cutoff values; generating, via the at least one processor using the at least one machine learning model and the historical data, at least one machine learning model test; receiving, at the computer system, new sample data associated a new sample; and executing, via the at least one processor, the at least one machine learning model test using the new sample data, resulting in a prediction of the new sample.

A method, comprising: receiving, at a computer system, historical data, the historical data comprising: a plurality of known samples; a binary outcome of each sample in the plurality of known samples; and variable data for each sample in the plurality of known samples, wherein the variable data comprises results for a plurality of variables; training a neural network using the historical data; identifying, via at least one processor of the computer system based on the historical data, a cutoff value for each variable in the plurality of variables, resulting in a plurality of cutoff values; generating, via the at least one processor using the plurality of cutoff values and the neural network, at least one combination test; receiving, at the computer system, new sample data associated a new sample; and executing, via the at least one processor, the at least one combination test using the new sample data, resulting in a prediction of the new sample.

A method, comprising: receiving, at a computer system, historical data, the historical data comprising: a plurality of known samples; a binary outcome of each sample in the plurality of known samples; and variable data for each sample in the plurality of known samples, wherein the variable data comprises results for a plurality of variables; identifying, via at least one processor, a cutoff value for each variable in the plurality of variables, resulting in a plurality of cutoff values; and generating individual variable tests for each variable based on the plurality of cutoff values.

A method, comprising: receiving, at a computer system, original historical data, the original historical data comprising: a plurality of known samples; a binary outcome of each sample in the plurality of known samples; and variable data for each sample in the plurality of known samples, wherein the variable data comprises results for a plurality of variables; identifying, via at least one processor of the computer system based on the historical data, a cutoff value for each variable in the plurality of variables, resulting in a plurality of cutoff values; generating, via the at least one processor using the plurality of cutoff values, at least one combination test; generating an ensemble test using the at least one combination test, wherein generation of the ensemble test comprises: creating a copy of the original historical data to be used during the generation of the ensemble test, the copy being filtered historical data; and iteratively selecting a combination test from within the at least one combination test, resulting in a selected test, wherein each time a selected test within the at least one combination test is selected for the ensemble test: the filtered historical data are iteratively filtered (to remove any samples now predicted as positive by the still being assembled ensemble that now includes that selected combination test); the filtered historical data are used to identify, via at least one processor of the computer system, a new cutoff value for each variable in the plurality of variables, resulting in a plurality of new cutoff values; generating, via the at least one processor of the computer system and using the plurality of new cutoff values, at least one new combination test; selecting, via the at least one processor from the original historical data at least one new combination test from among the at least one new combination test for inclusion in the ensemble test until completion of the ensemble test assembly, such that: each of a plurality of selected combination tests for the ensemble test have overlap below a predetermined value in true positive results with other tests within the plurality of selected combination tests; and receiving, at the computer system, new sample data associated a new sample; and executing, via the at least one processor, the ensemble test using the new sample data, resulting in a prediction of the new sample. Non-limiting examples of the predetermined value of overlap can be 2% overlap, 1% overlap, etc.

The method of any preceding clause, further comprising: automatically screening new sample data based on one or more of the individual variable tests, resulting in a prediction regarding the new sample data.

The method of any preceding clause, further comprising: creating, based on the individual variable tests, at least one combination test, the at least one combination test combining at least two of the individual variable tests.

The method of any preceding clause, wherein the at least one combination test comprises all possible combinations of the individual variable tests.

The method of any preceding clause, wherein each combination test within the at least one combination test are analyzed to identify which combination tests should be part of an ensemble test, the ensemble test comprising an ensemble of at least one selected combination test from within the at least one combination tests.

The method of any preceding clause, further comprising training at least one of a machine learning algorithm or a neural network using the historical data.

The method of any preceding clause, wherein the at least one combination tests comprise a combination of at least one individual test and at least one test based on the machine learning test or the neural network.

The method of any preceding clause, wherein each time a selected test within the plurality of tests is selected for the ensemble test: the historical data are iteratively filtered, resulting in filtered historical data, thereby removing any samples within the filtered historical data now predicted as positive by one or more selected combination tests for the ensemble test; the filtered historical data are used to identify, via at least one processor of the computer system, a new cutoff value for each variable in the plurality of variables, resulting in a plurality of new cutoff values; generating, via the at least one processor of the computer system and using the plurality of new cutoff values, at least one new combination test; selecting, via the at least one processor from the historical data, at least one new combination test from among the at least one new combination test for inclusion in the ensemble test until completion of the ensemble test, wherein each of a plurality of selected combination tests for the ensemble test have overlap below a predetermined value in true positive results with other tests within the plurality of selected combination tests; and receiving, at the computer system, new sample data associated a new sample; and executing, via the at least one processor, the ensemble test using the new sample data, resulting in a prediction of the new sample. Non-limiting examples of the predetermined value of overlap can include 1%, 2%, or any other number desired by a user.

The method of any preceding clause, wherein the identifying of the cutoff value for each variable further comprises: generating a Receiver Operating Characteristic (ROC) curve for each variable in the plurality of variables, the ROC curve plotting the binary outcome against the each variable, resulting in a plurality of ROC curves where each ROC curve in the plurality of ROC curves is associated with a variable in the plurality of variables; and identifying a value for the variable in the plurality of variables, the value corresponding to a highest ratio point of a True Positive Rate (TPR) over a False Positive Rate (FPR) within each ROC curve in the plurality of ROC curves, wherein the value is the cutoff value.

The method of any preceding clause, wherein the generating of the at least one combination test comprises: for each variable in the plurality of variables: combining the cutoff value for the each variable with at least one cutoff value for at least additional variable from the plurality of variables, resulting in combined cutoff values, wherein the combined cutoff values result in a combination test where each cutoff value in the combined cutoff values must be met for a positive result.

The method of any preceding clause, wherein the combined cutoff values result in combinations of at least three variables within the plurality of variables.

The method of any preceding clause, further comprising: for each combination test in the at least one combination test, calculating a True Positive Rate (TPR) using the combination test on the historical data, resulting in TPR results; identifying a highest scoring combination test within the at least one combination test based on the TPR results; and selecting the highest scoring combination test as one of at least one combination test to be performed on the new sample, wherein the generating of the prediction of the new sample uses the at least one combination to be performed.

The method of any preceding clause, wherein the selecting of the highest scoring combination test as one of the at least one combination test to be performed continues until a predetermined maximum number of combination tests to be performed is reached.

The method of any preceding clause, wherein if any result of the at least one combination test provides a positive result regarding the new sample, the new sample tests positive for a given condition.

The method of any preceding clause, wherein the at least one combination test are used to form an ensemble test.

The method of cany preceding clause, further comprising: generating an ensemble test, the ensemble test comprising one or more of the at least one combination test or a machine learning model test.

The method of any preceding clause, further comprising: generating, via the at least one processor using at least one machine learning model and the historical data, at least one machine learning model test; and generating, via the at least one processor using at least one of the at least one combination test or the at least one machine learning model test, a prediction of the new sample.

The method of any preceding clause, further comprising: generating an ensemble test, the ensemble test comprising the at least one of the at least one combination test or the at least one machine learning model test or a neural network test, wherein the prediction of the new sample is generated by executing the ensemble test on the data associated with the new sample.

The method of any preceding clause, wherein the at least one machine learning model comprises at least one of: a logistic regression model, a random forest model, a K-nearest neighbors model, or a gradient boosting model.

The method of any preceding clause, wherein a training dataset is recursively filtered.

The method of any preceding clause, further comprising creating a pool of candidate combination tests, where a combination test is considered positive for a given sample if and only if every variable within that combination test is positive (i.e., based upon if the cutoff threshold is exceeded).

The method of any preceding clause, wherein the identifying of the cutoff value for each variable further comprises: generating a Receiver Operating Characteristic (ROC) curve for each variable in the plurality of variables, the ROC curve plotting the binary outcome against the each variable, resulting in a plurality of ROC curves where each ROC curve in the plurality of ROC curves is associated with a variable in the plurality of variables; and identifying a value for the variable in the plurality of variables, the value corresponding to a highest ratio point of a True Positive Rate (TPR) over a False Positive Rate (FPR) within each ROC curve in the plurality of ROC curves, wherein the value is the cutoff value.

The method of any preceding clause, wherein the generating of the at least one combination test comprises: for each variable in the plurality of variables: combining the cutoff value for the each variable with at least one cutoff value for at least additional variable from the plurality of variables, resulting in combined cutoff values, wherein the combined cutoff values result in a combination test where each cutoff value in the combined cutoff values must be met for a positive result.

The method of any preceding clause, wherein the combined cutoff values result in combinations of at least three variables within the plurality of variables.

The method of any preceding clause, further comprising: for each combination test in the at least one combination test, calculating a True Positive Rate (TPR) using the combination test on the historical data, resulting in TPR results; identifying a highest scoring combination test within the at least one combination test based on the TPR results; and selecting the highest scoring combination test as one of at least one combination test to be performed on the new sample, wherein the generating of the prediction of the new sample uses the at least one combination to be performed.

The method of any preceding clause, wherein the selecting of the highest scoring combination test as one of the at least one combination test to be performed continues until a predetermined maximum number of combination tests to be performed is reached.

The method of any preceding clause, further comprising: performing a medical procedure based on the prediction.

The method of any preceding clause, wherein the at least one combination test are used as part of an ensemble test.

The method of any preceding clause, further comprising: generating an ensemble test, the ensemble test comprising at least one combination test and the at least one machine learning model test, wherein the prediction of the new sample is generated by executing the ensemble test on the data associated with the new sample.

The method of any preceding clause, wherein the at least one machine learning model comprises at least one of: a logistic regression model, a random forest model, a K-nearest neighbors model, a gradient boosting model, or a neural network model.

The method of any preceding clause, further comprising: performing a medical procedure based on the prediction.

The method of any preceding clause, wherein the medical procedure comprises at least one of a mammogram or a colonoscopy.

A system comprising: at least one processor; and a non-transitory computer-readable storage medium having instructions stored which, when executed by the at least one processor, cause the at least one processor to perform operations comprising: the method of any preceding clause.

A system comprising: at least one processor; and a non-transitory computer-readable storage medium having instructions stored which, when executed by the at least one processor, cause the at least one processor to perform operations comprising: receiving historical data, the historical data comprising: a plurality of known samples; a binary outcome of each sample in the plurality of known samples; and variable data for each sample in the plurality of known samples, wherein the variable data comprises results for a plurality of variables; identifying, based on the historical data, a cutoff value for each variable in the plurality of variables, resulting in a plurality of cutoff values; generating, using the plurality of cutoff values, at least one combination test; receiving data associated a new sample; and generating, using the at least one combination test, a prediction of the new sample.

A system comprising: at least one processor; and a non-transitory computer-readable storage medium having instructions stored which, when executed by the at least one processor, cause the at least one processor to perform operations comprising: receiving historical data, the historical data comprising: a plurality of known samples; a binary outcome of each sample in the plurality of known samples; and variable data for each sample in the plurality of known samples, wherein the variable data comprises results for a plurality of variables; training a neural network using the historical data; identifying, based on the historical data, a cutoff value for each variable in the plurality of variables, resulting in a plurality of cutoff values; generating, using the plurality of cutoff values and the neural network, at least one combination test; receiving new sample data associated a new sample; and executing the at least one combination test on the new sample data, resulting in a prediction of the new sample.

The system of any preceding clause, wherein the identifying of the cutoff value for each variable further comprises: generating a Receiver Operating Characteristic (ROC) curve for each variable in the plurality of variables, the ROC curve plotting the binary outcome against the each variable, resulting in a plurality of ROC curves where each ROC curve in the plurality of ROC curves is associated with a variable in the plurality of variables; and identifying a value for the variable in the plurality of variables, the value corresponding to a highest ratio point of a True Positive Rate (TPR) over a False Positive Rate (FPR) within each ROC curve in the plurality of ROC curves, wherein the value is the cutoff value.

The system of any preceding clause, wherein the generating of the at least one combination test comprises: for each variable in the plurality of variables: combining the cutoff value for the each variable with at least one cutoff value for at least additional variable from the plurality of variables, resulting in combined cutoff values, wherein the combined cutoff values result in a combination test where each cutoff value in the combined cutoff values must be met for a positive result.

The system of any preceding clause, wherein the combined cutoff values result in combinations of at least three variables within the plurality of variables.

The system of any preceding clause, the non-transitory computer-readable storage medium having additional instructions stored which, when executed by the at least one processor, cause the at least one processor to perform operations comprising: for each combination test in the at least one combination test, calculating a True Positive Rate (TPR) using the combination test on the historical data, resulting in TPR results; identifying a highest scoring combination test within the at least one combination test based on the TPR results; and selecting the highest scoring combination test as one of at least one combination test to be performed on the new sample, wherein the generating of the prediction of the new sample uses the at least one combination to be performed.

The system of any preceding clause, wherein the selecting of the highest scoring combination test as one of the at least one combination test to be performed continues until a predetermined maximum number of combination tests to be performed is reached.

The system of any preceding clause, wherein the identifying of the cutoff value for each variable further comprises: generating a Receiver Operating Characteristic (ROC) curve for each variable in the plurality of variables, the ROC curve plotting the binary outcome against the each variable, resulting in a plurality of ROC curves where each ROC curve in the plurality of ROC curves is associated with a variable in the plurality of variables; and identifying a value for the variable in the plurality of variables, the value corresponding to a highest ratio point of a True Positive Rate (TPR) over a False Positive Rate (FPR) within each ROC curve in the plurality of ROC curves, wherein the value is the cutoff value.

The system of any preceding clause, wherein the generating of the at least one combination test comprises: for each variable in the plurality of variables: combining the cutoff value for the each variable with at least one cutoff value for at least additional variable from the plurality of variables, resulting in combined cutoff values, wherein the combined cutoff values result in a combination test where each cutoff value in the combined cutoff values must be met for a positive result.

The system of any preceding clause, wherein the combined cutoff values result in combinations of at least three variables within the plurality of variables.

The system of any preceding clause, the non-transitory computer-readable storage medium having additional instructions stored which, when executed by the at least one processor, cause the at least one processor to perform operations comprising: for each combination test in the at least one combination test, calculating a True Positive Rate (TPR) using the combination test on the historical data, resulting in TPR results; calculating a Positive Predictive Value (PPV) using the combination test on the historical data, resulting in PPV results; identifying a highest scoring combination test within the at least one combination test based on the TPR results that also has PPV results above the user-specified minimum PPV; and selecting the highest scoring combination test as one of at least one combination test to be performed on the new sample, wherein the generating of the prediction of the new sample uses the at least one combination to be performed.

The system of any preceding clause, wherein the selecting of the highest scoring combination test as one of the at least one combination test to be performed continues until a predetermined maximum number of combination tests to be performed is reached.

A non-transitory computer-readable storage medium having instructions stored which, when executed by at least one processor, cause the at least one processor to perform operations comprising: the method of any preceding clause.

A non-transitory computer-readable storage medium having instructions stored which, when executed by at least one processor, cause the at least one processor to perform operations comprising: receiving historical data, the historical data comprising: a plurality of known samples; a binary outcome of each sample in the plurality of known samples; and variable data for each sample in the plurality of known samples, wherein the variable data comprises results for a plurality of variables; identifying, based on the historical data, a cutoff value for each variable in the plurality of variables, resulting in a plurality of cutoff values; generating, using the plurality of cutoff values, at least one combination test; receiving data associated a new sample; and generating, using the at least one combination test, a prediction of the new sample.

A non-transitory computer-readable storage medium having instructions stored which, when executed by at least one processor, cause the at least one processor to perform operations comprising: receiving historical data, the historical data comprising: a plurality of known samples; a binary outcome of each sample in the plurality of known samples; and variable data for each sample in the plurality of known samples, wherein the variable data comprises results for a plurality of variables; training a neural network using the historical data; identifying, based on the historical data, a cutoff value for each variable in the plurality of variables, resulting in a plurality of cutoff values; generating, using the plurality of cutoff values and the neural network, at least one combination test; receiving new sample data associated a new sample; and executing the at least one combination test using the new sample data, resulting in a prediction of the new sample.

The non-transitory computer-readable storage medium of any preceding clause, wherein the identifying of the cutoff value for each variable further comprises: generating a Receiver Operating Characteristic (ROC) curve for each variable in the plurality of variables, the ROC curve plotting the binary outcome against the each variable, resulting in a plurality of ROC curves where each ROC curve in the plurality of ROC curves is associated with a variable in the plurality of variables; and identifying a value for the variable in the plurality of variables, the value corresponding to a highest ratio point of a True Positive Rate (TPR) over a False Positive Rate (FPR) within each ROC curve in the plurality of ROC curves, wherein the value is the cutoff value.

The non-transitory computer-readable storage medium of any preceding clause, wherein the generating of the at least one combination test comprises: for each variable in the plurality of variables: combining the cutoff value for the each variable with at least one cutoff value for at least additional variable from the plurality of variables, resulting in combined cutoff values, wherein the combined cutoff values result in a combination test where each cutoff value in the combined cutoff values must be met for a positive result.

The non-transitory computer-readable storage medium of any preceding clause, wherein the combined cutoff values result in combinations of at least three variables within the plurality of variables.

The non-transitory computer-readable storage medium of any preceding clause, having additional instructions stored which, when executed by the at least one processor, cause the at least one processor to perform operations comprising: for each combination test in the at least one combination test, calculating a True Positive Rate (TPR) using the combination test on the historical data, resulting in TPR results; calculating a Positive Predictive Value (PPV) using the combination test on the historical data, resulting in PPV results; identifying a highest scoring combination test within the at least one combination test based on the TPR results that also has PPV results above the user-specified minimum PPV; and selecting the highest scoring combination test as one of at least one combination test to be performed on the new sample, wherein the generating of the prediction of the new sample uses the at least one combination to be performed.

The non-transitory computer-readable storage medium of any preceding clause, wherein the selecting of the highest scoring combination test as one of the at least one combination test to be performed continues until a predetermined maximum number of combination tests to be performed is reached.

The non-transitory computer-readable storage medium of any preceding clause, wherein the identifying of the cutoff value for each variable further comprises: generating a Receiver Operating Characteristic (ROC) curve for each variable in the plurality of variables, the ROC curve plotting the binary outcome against the each variable, resulting in a plurality of ROC curves where each ROC curve in the plurality of ROC curves is associated with a variable in the plurality of variables; and identifying a value for the variable in the plurality of variables, the value corresponding to a highest ratio point of a True Positive Rate (TPR) over a False Positive Rate (FPR) within each ROC curve in the plurality of ROC curves, wherein the value is the cutoff value.

The non-transitory computer-readable storage medium of any preceding clause, wherein the generating of the at least one combination test comprises: for each variable in the plurality of variables: combining the cutoff value for the each variable with at least one cutoff value for at least additional variable from the plurality of variables, resulting in combined cutoff values, wherein the combined cutoff values result in a combination test where each cutoff value in the combined cutoff values must be met for a positive result.

The non-transitory computer-readable storage medium of any preceding clause, wherein the combined cutoff values result in combinations of at least three variables within the plurality of variables.

The non-transitory computer-readable storage medium of any preceding clause, having additional instructions stored which, when executed by the at least one processor, cause the at least one processor to perform operations comprising: for each combination test in the at least one combination test, calculating a True Positive Rate (TPR) using the combination test on the historical data, resulting in TPR results; identifying a highest scoring combination test within the at least one combination test based on the TPR results; and selecting the highest scoring combination test as one of at least one combination test to be performed on the new sample, wherein the generating of the prediction of the new sample uses the at least one combination to be performed.

The non-transitory computer-readable storage medium of any preceding clause, wherein the selecting of the highest scoring combination test as one of the at least one combination test to be performed continues until a predetermined maximum number of combination tests to be performed is reached.

We claim:

1. A method for improving predictions within a sample, comprising:
receiving, at a computer system, historical data, the historical data comprising:
a plurality of known samples;
a binary outcome of each sample in the plurality of known samples; and
variable data for each sample in the plurality of known samples,
wherein the variable data comprises results for a plurality of variables;
training at least one neural network using the historical data;
identifying, via at least one processor of the computer system based on the historical data, a cutoff value for each variable in the plurality of variables, resulting in a plurality of cutoff values;
generating, via the at least one processor using the plurality of cutoff values and the at least one neural network, at least one combination test;
receiving, at the computer system, new sample data associated a new sample; and
executing, via the at least one processor, the at least one combination test using the new sample data, resulting in a prediction of the new sample.

2. The method of claim 1, wherein the identifying of the cutoff value for each variable further comprises:
generating a Receiver Operating Characteristic (ROC) curve for each variable in the plurality of variables, the ROC curve plotting the binary outcome against the each variable, resulting in a plurality of ROC curves where each ROC curve in the plurality of ROC curves is associated with a variable in the plurality of variables; and
identifying a value for the variable in the plurality of variables, the value corresponding to a highest ratio point of a True Positive Rate (TPR) over a False Positive Rate (FPR) within each ROC curve in the plurality of ROC curves,
wherein the value is the cutoff value.

3. The method of claim 1, wherein the generating of the at least one combination test comprises:
for each variable in the plurality of variables:
combining the cutoff value for the each variable with at least one cutoff value for at least additional variable from the plurality of variables, resulting in combined cutoff values, wherein the combined cutoff values result in a combination test where each cutoff value in the combined cutoff values must be met for a positive result.

4. The method of claim 3, wherein the combined cutoff values result in combinations of at least three variables within the plurality of variables.

5. The method of claim 1, further comprising:
for each combination test in the at least one combination test, calculating a True Positive Rate (TPR) using the combination test on the historical data, resulting in TPR results;
identifying a highest scoring combination test within the at least one combination test based on the TPR results; and
selecting the highest scoring combination test as one of at least one combination test to be performed on the new sample,
wherein the generating of the prediction of the new sample uses the at least one combination to be performed.

6. The method of claim 5, wherein the selecting of the highest scoring combination test as one of the at least one combination test to be performed continues until a predetermined maximum number of combination tests to be performed is reached.

7. The method of claim 1, further comprising:
performing a medical procedure based on the prediction.

8. The method of claim 1, wherein the at least one combination test is used as part of an ensemble test which uses a plurality of tests selected from the at least one combination test or the at least one neural network or at least one machine learning model,
wherein each time a selected test within the plurality of tests is selected for the ensemble test:
the historical data are iteratively filtered, resulting in filtered historical data, thereby removing any samples within the filtered historical data now predicted as positive by one or more selected combination tests for the ensemble test;
the filtered historical data are used to identify, via at least one processor of the computer system, a new cutoff value for each variable in the plurality of variables, resulting in a plurality of new cutoff values;
generating, via the at least one processor of the computer system and using the plurality of new cutoff values, at least one new combination test;
selecting, via the at least one processor from the historical data, at least one new combination test from among the at least one new combination test for inclusion in the ensemble test until completion of the ensemble test,
wherein each of a plurality of selected combination tests for the ensemble test have overlap below a predetermined value in true positive results with other tests within the plurality of selected combination tests; and
receiving, at the computer system, new sample data associated a new sample; and
executing, via the at least one processor, the ensemble test using the new sample data, resulting in a prediction of the new sample.

9. A system comprising:
at least one processor; and
a non-transitory computer-readable storage medium having instructions stored which, when executed by the at least one processor, cause the at least one processor to perform operations comprising:
receiving historical data, the historical data comprising:
a plurality of known samples;
a binary outcome of each sample in the plurality of known samples; and
variable data for each sample in the plurality of known samples,
wherein the variable data comprises results for a plurality of variables;
training a neural network using the historical data;
identifying, based on the historical data, a cutoff value for each variable in the plurality of variables, resulting in a plurality of cutoff values;
generating, using the plurality of cutoff values and the neural network, at least one combination test;
receiving new sample data associated a new sample; and
executing the at least one combination test on the new sample data, resulting in a prediction of the new sample.

10. The system of claim 9, wherein the identifying of the cutoff value for each variable further comprises:
generating a Receiver Operating Characteristic (ROC) curve for each variable in the plurality of variables, the ROC curve plotting the binary outcome against the each variable, resulting in a plurality of ROC curves where each ROC curve in the plurality of ROC curves is associated with a variable in the plurality of variables; and
identifying a value for the variable in the plurality of variables, the value corresponding to a highest ratio point of a True Positive Rate (TPR) over a False Positive Rate (FPR) within each ROC curve in the plurality of ROC curves,
wherein the value is the cutoff value.

11. The system of claim 9, wherein the generating of the at least one combination test comprises:
for each variable in the plurality of variables:
combining the cutoff value for the each variable with at least one cutoff value for at least additional variable from the plurality of variables, resulting in combined cutoff values, wherein the combined cutoff values result in a combination test where each cutoff value in the combined cutoff values must be met for a positive result.

12. The system of claim 11, wherein the combined cutoff values result in combinations of at least three variables within the plurality of variables.

13. The system of claim 9, the non-transitory computer-readable storage medium having additional instructions stored which, when executed by the at least one processor, cause the at least one processor to perform operations comprising:
for each combination test in the at least one combination test, calculating a True Positive Rate (TPR) using the combination test on the historical data, resulting in TPR results;
identifying a highest scoring combination test within the at least one combination test based on the TPR results; and
selecting the highest scoring combination test as one of at least one combination test to be performed on the new sample,
wherein the generating of the prediction of the new sample uses the at least one combination to be performed.

14. The system of claim 13, wherein the selecting of the highest scoring combination test as one of the at least one combination test to be performed continues until a predetermined maximum number of combination tests to be performed is reached.

15. A non-transitory computer-readable storage medium having instructions stored which, when executed by at least one processor, cause the at least one processor to perform operations comprising:
receiving historical data, the historical data comprising:
a plurality of known samples;
a binary outcome of each sample in the plurality of known samples; and
variable data for each sample in the plurality of known samples,
wherein the variable data comprises results for a plurality of variables;
training a neural network using the historical data;
identifying, based on the historical data, a cutoff value for each variable in the plurality of variables, resulting in a plurality of cutoff values;
generating, using the plurality of cutoff values and the neural network, at least one combination test;
receiving new sample data associated a new sample; and
executing the at least one combination test using the new sample data, resulting in a prediction of the new sample.

16. The non-transitory computer-readable storage medium of claim 15, wherein the identifying of the cutoff value for each variable further comprises:
generating a Receiver Operating Characteristic (ROC) curve for each variable in the plurality of variables, the ROC curve plotting the binary outcome against the each variable, resulting in a plurality of ROC curves where each ROC curve in the plurality of ROC curves is associated with a variable in the plurality of variables; and
identifying a value for the variable in the plurality of variables, the value corresponding to a highest ratio point of a True Positive Rate (TPR) over a False Positive Rate (FPR) within each ROC curve in the plurality of ROC curves,
wherein the value is the cutoff value.

17. The non-transitory computer-readable storage medium of claim 15, wherein the generating of the at least one combination test comprises:
for each variable in the plurality of variables:
combining the cutoff value for the each variable with at least one cutoff value for at least additional variable from the plurality of variables, resulting in combined cutoff values, wherein the combined cutoff values result in a combination test where each cutoff value in the combined cutoff values must be met for a positive result.

18. The non-transitory computer-readable storage medium of claim 17, wherein the combined cutoff values result in combinations of at least three variables within the plurality of variables.

19. The non-transitory computer-readable storage medium of claim 15, having additional instructions stored which, when executed by the at least one processor, cause the at least one processor to perform operations comprising:
for each combination test in the at least one combination test, calculating a True Positive Rate (TPR) using the combination test on the historical data, resulting in TPR results;
identifying a highest scoring combination test within the at least one combination test based on the TPR results; and
selecting the highest scoring combination test as one of at least one combination test to be performed on the new sample,
wherein the generating of the prediction of the new sample uses the at least one combination to be performed.

20. The non-transitory computer-readable storage medium of claim 19, wherein the selecting of the highest scoring combination test as one of the at least one combination test to be performed continues until a predetermined maximum number of combination tests to be performed is reached.

21. A method comprising:
receiving, at a computer system, historical data, the historical data comprising:
a plurality of known samples;
a binary outcome of each sample in the plurality of known samples;
variable data for each sample in the plurality of known samples,
wherein the variable data comprises results for a plurality of variables;
training a neural network using the historical data;
identifying, via at least one processor of the computer system and by running a screening test, a subset of samples from the historical data which the screening test identifies as positive;
identifying, via the at least one processor and using the subset of samples, a cutoff value for each variable in the plurality of variables, resulting in a plurality of cutoff values;
generating, via the at least one processor using the plurality of cutoff values and the neural network, at least one confirmation combination test;
receiving, at the computer system, new sample data associated with a new sample;
executing, via the at least one processor using the data, the screening test on the new sample data, resulting in a screening prediction of the new sample; and
executing, via the at least one processor using the data, the confirmation combination test on the new sample data, resulting in a confirmation prediction regarding the new sample.

22. A method, comprising:
receiving, at a computer system, historical data, the historical data comprising:
a plurality of known samples;
a binary outcome of each sample in the plurality of known samples; and
variable data for each sample in the plurality of known samples,
wherein the variable data comprises results for a plurality of variables;
training at least one machine learning model using the historical data;
identifying, via at least one processor of the computer system based on the historical data, a cutoff value for each variable in the plurality of variables, resulting in a plurality of cutoff values;
generating, via the at least one processor using the at least one machine learning model and the historical data, at least one machine learning model test;
receiving, at the computer system, new sample data associated a new sample; and
executing, via the at least one processor, the at least one machine learning model test using the new sample data, resulting in a prediction of the new sample.

23. The method of claim 22, further comprising:
generating an ensemble test, the ensemble test comprising at least one combination test and the at least one machine learning model test,
wherein the prediction of the new sample is generated by executing the ensemble test on the data associated with the new sample.

24. The method of claim 22, wherein the at least one machine learning model comprises at least one of: a logistic regression model, a random forest model, a K-nearest neighbors model, a gradient boosting model, or a neural network model.

25. The method of claim 22, further comprising:
performing a medical procedure based on the prediction.

26. The method of claim 25, wherein the medical procedure comprises at least one of a mammogram or a colonoscopy.
